SEQUENCE LISTING (24 sequences)

SEQ ID NO: 1 - DNA, 24 bp, Artificial
  synthetic primer
gctggcagtggggagcacagtgca

SEQ ID NO: 2 - DNA, 24 bp, Artificial
  synthetic primer
agggcaggaaaggcagcagcactt

SEQ ID NO: 3 - DNA, 24 bp, Artificial
  synthetic primer
aaggcatgattcattcacctgaga

SEQ ID NO: 4 - DNA, 24 bp, Artificial
  synthetic primer
agggcaggaaaggcagcagcactt

SEQ ID NO: 5 - DNA, 20 bp, Artificial
  synthetic primer
gtcaagaaggcgatagaagg

SEQ ID NO: 6 - DNA, 23 bp, Artificial
  synthetic primer
ttgaacaagatggattgcacgca

SEQ ID NO: 7 - DNA, 20 bp, Artificial
  synthetic primer
tgcagctgacacttttacgg

SEQ ID NO: 8 - DNA, 20 bp, Artificial
  synthetic primer
ttcccttggatttcagcttg

SEQ ID NO: 9 - DNA, 20 bp, Artificial
  synthetic primer
tcaagattggactgctggtg

SEQ ID NO: 10 - DNA, 20 bp, Artificial
  synthetic primer
aggtttggtctggaggaggt

SEQ ID NO: 11 - DNA, 20 bp, Artificial
  synthetic primer
tcaatggcaacaactgggta

SEQ ID NO: 12 - DNA, 20 bp, Artificial
  synthetic primer
atgacccacactatgccaca

SEQ ID NO: 13 - DNA, 20 bp, Artificial
  synthetic primer
taccacaggcattgtgatgg

SEQ ID NO: 14 - DNA, 20 bp, Artificial
  synthetic primer
tttgatgtcacgcacgattt

SEQ ID NO: 15 - DNA, 20 bp, Artificial
  synthetic primer
gggactagcatgaacggcta

SEQ ID NO: 16 - DNA, 20 bp, Artificial
  synthetic primer
ccccaaccgaaatttcaaac

SEQ ID NO: 17 - DNA, 20 bp, Artificial
  synthetic primer
ctgagcgggaatagtgggta

SEQ ID NO: 18 - DNA, 20 bp, Artificial
  synthetic primer
tggggctccgattattagtg

SEQ ID NO: 19 - DNA, 20 bp, Artificial
  synthetic primer
acgtccttccatgaggacaa

SEQ ID NO: 20 - DNA, 20 bp, Artificial
  synthetic primer
gaggtgaacgattgctaggg

SEQ ID NO: 21 - DNA, 21 bp, Artificial
  siRNA
gguugacuucacagaagaatt

SEQ ID NO: 22 - DNA, 21 bp, Artificial
  siRNA
uucuucugugaagucaacctt

SEQ ID NO: 23 - DNA, 21 bp, Artificial
  siRNA
ggaauucaggaaauauuuctt

SEQ ID NO: 24 - DNA, 21 bp, Artificial
  siRNA
gaaauauuuccugaauucctg

(19) United States
(12) Patent Application Publication
(10) Pub. No.: US 2010/0169995 A1
(43) Pub. Date: Jul. 1, 2010

(54) TARGETED GENE DISRUPTIONS IN THE IQGAP2 GENE

(75) Inventors: Valentina Schmidt, Port Jefferson, NY (US); Wadie F. Bahou, Setauket, NY (US)

Correspondence Address: KENYON & KENYON LLP, ONE BROADWAY, NEW YORK, NY, 10004, US

(73) Assignee: The Research Foundation of State University of New York, Albany, NY (US)

(21) Appl. No.: 12/640,876

(22) Filed: Dec. 17, 2009

Related U.S. Application Data
(60) Provisional application No. 61/138,276, filed on Dec. 17, 2008.

(51) Int. Cl. A01K 67/027; A01K 67/033; A01K 67/00; C12N 5/10; C12N 15/74; G01N 33/567; A61K 31/713; A61P 35/00

(52) U.S. Cl. 800/18; 800/8; 800/13; 435/325; 435/354; 435/320.1; 800/21; 435/7.21; 514/44 R

(57) ABSTRACT

The invention provides a non-human mammal or a cell line that has a targeted gene disruption in an endogenous Iqgap2 gene. The invention also provides methods of identifying a compound as a therapeutic agent for the treatment of hepatocellular carcinoma and methods of treating or preventing hepatocellular carcinoma.

### CROSS REFERENCE TO RELATED APPLICATIONS

This application claims priority to U.S. Application No. 61/136,276, filed Dec. 17, 2008, which is incorporated herein by reference in its entirety.

### STATEMENT OF GOVERNMENT INTEREST

The invention was made with United States Government support under Grant Nos. HL04941 and DK062040 awarded by the National Institutes of Health. The Government has certain rights in this invention.

### FIELD OF THE INVENTION

The invention relates to a non-human mammal that has a targeted gene disruption in an endogenous Iqgap2 gene. In another embodiment, the invention includes cell lines that have a targeted gene disruption in an endogenous Iqgap2 gene. The invention also provides methods of identifying a compound as a therapeutic agent for the treatment of hepatocellular carcinoma and methods of treating or preventing hepatocellular carcinoma.

### BACKGROUND OF THE INVENTION

Hepatocellular carcinoma (HCC) accounts for over 80% of all human liver cancer, and is responsible for between 500,000 and 1 million worldwide deaths annually. Predisposing etiologies for HCC include chronic hepatitis B and C virus infections, aflatoxin B1, chronic alcohol consumption, or any hepatic disease associated with cirrhosis. The diverse etiology of HCC results in its considerable genomic heterogeneity. In 2005, HCC incidence was estimated as 667,000 cases globally and 17,550 cases in the United States. It is predicted that in the U.S. the number of cases of HCC will continue to grow by 81% by the year 2020. Overall survival of patients with HCC has not improved in the last two decades. Potentially curative therapies include resection, transplantation and percutaneous ablation and can be applied to only 30% to 40% patients at early stages of disease. Due to limited donor liver availability, only a small number of potential candidates will have access to liver transplantation. Most patients with HCC are diagnosed in later stages, when the disease is no longer amenable to potentially curative interventions. Several treatments for HCC have been tested in the past, including antiestrogens, interferon, anti-androgens, internal radiation and cytotoxic chemotherapy. In all studies, no survival benefit was demonstrated, and the rate of side effects undermining quality of life was high.

IQGAP1 and IQGAP2 are conserved homologues of an extended family of putative GTPase activating proteins largely studied in mammals as scaffolding proteins that have the ability to integrate intracellular signals with cytoskeletal membrane events. To date, IQGAP1 remains the best-characterized member of the family. IQGAPs are capable of binding F-actin, calmodulin, and GTPases cdc42 and rac1.

IQGAP2 is a primarily liver-specific protein (˜180 kDa), with moderate levels of expression in human platelets, while IQGAP1 is expressed broadly. Targeted disruption of the murine Iqgap1 gene identified a phenotype restricted to gastric mucosal hyperplasia. IQGAP1 has been shown to be involved in the control of cell adhesion and migration, and has been implicated in the promotion of invasiveness of various cancers, breast and gastrointestinal cancers in particular. IQGAP2 has also been demonstrated to function as a unique scaffolding protein linking thrombin activation to platelet cytoskeletal actin assembly and reorganization.

### SUMMARY OF THE INVENTION

It has been discovered that IQGAP2 functions as a novel tumor suppressor gene linked to the development of neoplasms and tumors. In a first aspect, the invention features a non-human mammal having a targeted gene disruption in an endogenous Iqgap2 gene. Embodiments of the invention may include one or more of the following features. In one embodiment the non-human mammal is homozygous for the targeted gene disruption. In another embodiment the non-human mammal with a targeted gene disruption in an endogenous Iqgap2 gene, exhibits an age-dependent increase in apoptosis of hepatocytes and/or exhibits an age-dependent increase in hepatocellular carcinoma.

It has also been discovered that IQGAP2 and IQGAP1 expression are related. Accordingly, in another embodiment the non-human mammal with a targeted gene disruption in an endogenous Iqgap2 gene also has a null allele of the endogenous Iqgap1 gene. In one embodiment the non-human mammal with a targeted gene disruption in an endogenous Iqgap2 gene is homozygous for a null allele of the endogenous Iqgap1 gene. In one embodiment the null allele is a targeted disruption in an endogenous Iqgap1 gene. In one embodiment, the non-human mammal with targeted gene disruptions in both Iqgap1 and Iqgap2 exhibits a reduction in age-dependent apoptosis or hepatocellular carcinoma associated with reduced Iqgap2 levels. In yet another embodiment, the non-human mammal with a targeted gene disruption in an endogenous Iqgap2 gene overexpresses Iqgap1.

In one embodiment the non-human mammal with a targeted gene disruption in an endogenous Iqgap2 gene is a mouse. In another embodiment, the non-human mammal is a rat.

In one embodiment the invention provides a cell isolated from the non-human mammal with a targeted gene disruption in an endogenous Iqgap2 gene. In one embodiment the cell is a hepatocyte, fibroblast, myocyte, adipocyte, lymphocyte, megacaryocyte, or platelet. In one embodiment the cell overexpresses Iqgap1. In one embodiment the invention provide a cell line derived from the non-human mammal with a targeted gene disruption in an endogenous Iqgap2 gene.

In general, in another aspect, the invention features a non-human mammal, which comprises a transgene that encodes an RNA that inhibits expression of the endogenous Iqgap2 gene. The RNA expressed from the transgene can be an antisense RNA, or a siRNA. In one embodiment the transgene that encodes an RNA that inhibits expression of the endogenous Iqgap2 gene contains a liver-specific promoter. In one embodiment the non-human mammal with a transgene that encodes an RNA that inhibits expression of the endogenous Iqgap2 gene, exhibits an age-dependent increase in apoptosis of hepatocytes and/or exhibits an age-dependent increase in hepatocellular carcinoma.

In one embodiment the non-human mammal, which comprises a transgene that encodes an RNA that inhibits expression of the endogenous Iqgap2 gene has a null allele of the endogenous Iqgap1 gene. In one embodiment the non-human mammal is homozygous for the null allele of the endogenous Iqgap1 gene. In one embodiment the null allele is a targeted disruption in an endogenous Iqgap1 gene. In one embodiment, the non-human mammal that contains both a transgene that encodes an RNA that inhibits expression of the endogenous Iqgap2 gene and a targeted gene disruption in Iqgap1 exhibits a reduction in age-dependent apoptosis or hepatocellular carcinoma associated with reduced Iqgap2 levels.

In one embodiment the invention provides a cell isolated from the non-human mammal with a transgene that encodes an RNA that inhibits expression of the endogenous Iqgap2 gene. In one embodiment the cell is a hepatocyte, fibroblast, myocyte, adipocyte, lymphocyte, megacaryocyte, or platelet. In one embodiment the cell overexpresses Iqgap1. In one embodiment, the invention provide a cell line derived from the non-human mammal with a transgene that encodes an RNA that inhibits expression of the endogenous Iqgap2 gene.

In general, in another aspect, the invention features a non-human mammal, which contains a transgene that expresses the Iqgap1 gene. In one embodiment, the Iqgap1 gene is overexpressed. In one embodiment, the Iqgap1 gene is ectopically expressed. In one embodiment the transgene that expresses the Iqgap1 gene contains a liver-specific promoter. In one embodiment the non-human mammal, which contains a transgene that overexpresses the Iqgap1 gene, exhibits an age-dependent increase in apoptosis of hepatocytes and/or exhibits an age-dependent increase in hepatocellular carcinoma.

In one embodiment the invention provides a cell isolated from the non-human mammal which contains a transgene that overexpresses the Iqgap1 gene. In one embodiment the cell is a hepatocyte, fibroblast, myocyte, adipocyte, lymphocyte, megacaryocyte, or platelet. In one embodiment, the invention provide a cell line derived from the non-human mammal which contains a transgene that overexpresses the Iqgap1 gene.

In general, in another aspect, the invention features a cell line which comprises an inactivated endogenous Iqgap2 gene. In one embodiment, the cell line is homozygous for the inactivated endogenous Iqgap2 gene. In one embodiment the endogenous Iqgap2 gene is inactivated by a targeted disruption. In another embodiment the endogenous Iqgap2 gene is inactivated by a small interfering RNA, microRNA, or anti-sense RNA. In one embodiment the cell line which comprises an inactivated endogenous Iqgap2 gene does not express Iqgap1. In one embodiment the genome of the cell line also contains a null allele of the endogenous Iqgap1 gene. In one embodiment the cell line is an embryonic stem cell line.

In general, in another aspect, the invention features a targeting construct having a first polynucleotide sequence homologous to a first portion of an endogenous Iqgap2 gene, a second polynucleotide sequence homologous to a second portion of the endogenous Iqgap2 gene, and a selectable marker located between the first and second polynucleotide sequences. In one embodiment the Iqgap2 gene in the targeting construct is a mouse gene.

In general, in another aspect, the invention features a method of producing a transgenic non-human mammal having a disruption in an endogenous Iqgap2 gene, comprising: a) introducing a targeting construct capable of disrupting the endogenous Iqgap2 gene into an embryonic stem cell of the mammal; b) selecting an embryonic stem cell that has undergone homologous recombination c) introducing the recombinant embryonic stem cell into a blastocyst; d) implanting the blastocyst into the mammal, wherein the mammal gives birth to a chimeric mammal; and e) breeding the chimeric mammal to produce the transgenic mammal, wherein where the disruption is homozygous, the transgenic mammal lacks production of a functional IQGAP2 protein. In one embodiment of this method the Iqgap1 gene is overexpressed in hepatocytes of the transgenic mammal.

In another aspect, the invention features a method of identifying a compound as a therapeutic agent for the treatment of hepatocellular carcinoma, comprising contacting the compound with a test cell that underexpresses Iqgap2, wherein Iqgap1 is overexpressed in response to Iqgap2 underexpression, and identifying the compound as a therapeutic agent for treatment of hepatocellular carcinoma if IQGAP1 activity is reduced in response to the compound. In one embodiment the test cell is a hepatocyte. In another embodiment, the IQGAP1 activity is evaluated by observing the level of Iqgap1 expression using an Iqgap1 reporter gene construct.

In another aspect, the invention features a method of identifying a compound as a therapeutic agent for the treatment of hepatocellular carcinoma, comprising: contacting the compound with a test cell that overexpresses Iqgap1, and identifying the compound as a therapeutic agent for treatment of hepatocellular carcinoma if IQGAP1 activity is reduced in response to the compound. In one embodiment, the test cell overexpresses human Iqgap1. In one embodiment the test cell is a hepatocyte. In another embodiment, the IQGAP1 activity is evaluated by observing the level of Iqgap1 expression using an Iqgap1 reporter gene construct.

In another aspect, the invention features a method of identifying a compound as a therapeutic agent for the treatment of hepatocellular carcinoma, comprising providing a non-human mammal which comprises a targeted gene disruption in an endogenous Iqgap2 gene and exhibits an age-dependent increase in apoptosis of hepatocytes, administering the compound to the non-human mammal, and identifying the compound as a therapeutic agent if it reduces the age-dependent increase in apoptosis.

In another aspect, the invention features a method of identifying a compound as a therapeutic agent for the treatment of hepatocellular carcinoma, comprising providing a non-human mammal which comprises a targeted gene disruption in an endogenous Iqgap2 gene and exhibits an age-dependent increase in hepatocellular carcinoma, administering the compound to the non-human mammal, and identifying the compound as a therapeutic agent if it reduces the age-dependent increase in hepatocellular carcinoma.

In another aspect, the invention features a model of human hepatocellular carcinoma comprising hepatocytes in which Iqgap2 is under-expressed. In one embodiment the reduction in the expression of Iqgap2 is accomplished by creating a null allele of the endogenous Iqgap2 gene. In another embodiment, the null allele of the endogenous Iqgap2 is homozygous.

In general, in another aspect, the invention features a method of treating or preventing hepatocellular carcinoma comprising administering an agent that decreases Iqgap1 function in hepatocytes. In one embodiment, the agent that decreases Iqgap1 function is a siRNA. In another embodiment the agent that decreases Iqgap1 function is a synthetic blocking peptide. In another embodiment the synthetic blocking peptide is directed against an IQGAP1 domain such as, for example, an actin-binding calponin homology domain, a SH3-mimicking domain, a calmodulin-binding domain, or a GTPase-binding domain.

In another aspect, the invention features a method of treating or preventing hepatocellular carcinoma by administering an agent that enhances Iqgap2 function in hepatocytes. In one embodiment the agent comprises a vector that expresses Iqgap2. In another embodiment the Iqgap2 is expressed from a liver-specific promoter. The liver specific promoter may be from the genes: albumin, α1-antitrypsin (AAT), phosphoenolpyruvate carboxykinase (PEPCK) and fatty acid binding protein (FABP). In one embodiment the agent comprises a methylation inhibitor. The methylation inhibitor may be a nucleoside inhibitor or a non-nucleoside inhibitor. Nucleoside inhibitors may include 5-azacytidine, 5-aza-2′-deoxy-cytidine, zebularine, and 5-fluoro-2′-deoxycytidine. The non-nucleoside inhibitor may include procainamide and hydralazine.

### BRIEF DESCRIPTION OF THE FIGURES

FIG. 1. Schema depicting the targeting construct and the Iqgap2 wild-type and null alleles, resulting in the replacement of a 36-kb genomic fragment (corresponding to amino acid residues Ala705 to Lys1306) with the PGK-Neo cassette. The deleted fragment encompasses a portion of the IQ2 (Gln696-to-Gly725), IQ3 (Asn726-to-Asp755), and IQ4 (His756-to-Pro785) domains and the GRD (Gln891 to Val1189). Selected numbered exons are depicted by rectangles, and the relative positions of oligonucleotides and the ³²P-radiolabeled probe used for allele identification are shown. A, ApaI; B, BamHI; X, XbaI.

FIG. 2. Southern blot analysis was performed using 10 μg of ApaI-digested genomic DNA isolated from various ES clones (Panel A) or Iqgap2^{-/-} (−/−), Iqgap2^{+/-} (+/−), or wild-type (+/+) mice (Panel B) specifically generated from ES clone no. 57, and DNA was probed with the ³²P-radiolabeled 500-bp genomic fragment depicted in FIG. 1. The 4-kb wild-type and 12-kb mutant alleles are evident.

FIG. 3. qRT-PCR was completed using total RNA isolated from livers of 2-month-old wild-type, Iqgap1^{-/-}, or Iqgap2^{-/-} mice and primers specific for the β-actin gene, Iqgap1, Iqgap2, and Par3 (known to be encompassed within the Iqgap2 gene).

FIG. 4. Total protein lysates (10 μg per lane) isolated from organs of 2-month-old Iqgap2^{-/-} (−/−) or Iqgap2^{-/-} (+/−) mice or wild-type (+/+) littermate controls were size fractionated by 4 to 15% SDS-PAGE, and IQGAP1 and IQGAP2 expression was assessed by immunoblotting. Note the absence of IQGAP2 in all Iqgap2^{-/-} organs, with a modest reciprocal increase in IQGAP1 in Iqgap2^{-/-} livers only.

FIG. 5. Age-dependent hepatopathy and mitochondrial damage in Iqgap2^{-/-} mice. (A) Sera from mice in three age groups (1, 4, and 8 months old; n=10 mice per age group) were analyzed for the quantification of AST. (B) Liver sections (n=3 per age group) were analyzed for apoptotic cells by TUNEL staining Results shown in panels A and B are means±SEM; statistically significant P values are shown.

FIG. 6. Transmission electron micrographs from livers and skeletal muscles of 12-month-old Iqgap2^{-/-} mice (low and high magnifications) (A) or wild-type mice (high magnification) (B) demonstrate an edematous mitochondrial matrix with collapsed cristae (solid arrows) restricted to Iqgap2^{-/-} mitochondria; hollow arrows indicate normal cristae. Note the normal architecture of mitochondria from skeletal muscles of both genotypes. Structural defects are representative of those seen in 8- and 12-month-old mice (n=3 per group), with comparable but less-extensive defects seen in 4-month-old Iqgap2^{-/-} mice. Size bars represent 2 μm (magnification, ×4,800 [A, top left panel]), 500 nm (magnification, ×18,500 [A, top right panel]), and 200 nm (magnification, ×49,000 [A, bottom panels, and B]). The white box denotes an Iqgap2^{-/-} mitochondrion shown magnified at ×49,000.

FIG. 7. Mitochondrial characterization of Iqgap2^{-/-} mice. (A) qRT-PCR was completed using total liver RNA from 12-month-old Iqgap2^{-/-} mice or age-matched wild-type controls. 16S, 16S subunit rRNA; COX, cytochrome c oxidase subunit 1; Cyt b, cytochrome b. The bar graphs show means±standard deviations of results from triplicate wells, normalized using β-actin; the results shown represent one complete set of experiments, repeated on two occasions. (B) Immunoblot analysis of liver and skeletal muscle lysates (10 μg/lane) from wild-type (+/+) and Iqgap2^{-/-} (−/−) mice using antibodies specific to complex I, complex II, prohibitin, and cytochrome c.

FIG. 8. Flow cytometry analysis of mitochondria (equivalent to 300×g of protein/sample) isolated from hepatocytes from 8-month-old Iqgap2^{-/-} or age-matched wild-type mice, loaded with tetramethylrhodamine ethyl ester, and incubated with the specified concentrations of valinomycin (A) or CaCl₂ (B). For all experiments, the percentage of dye retention was determined from the geometric mean for 10,000 events, with 100% representing the baseline fluorescence of intact mitochondria. Results are the means±SEM from two distinct experiments.

FIG. 9. Gross morphological evidence of HCC in the liver of a 2-year-old Iqgap2^{-/-} mouse compared to the liver of an age-matched wild-type control; asterisks denote multiple large, vascularized nodules. The scale bars correspond to 1 cm.

FIG. 10. (A) Representative hematoxylin- and eosin-stained sections from wild-type or Iqgap2^{-/-} livers demonstrating HCC in Iqgap2^{-/-} mice. The spiked line (right upper panel) delineates the border between normal (N) and tumor (T) tissue. Scale bars correspond to 150 μm (top panels) and 50 μm (bottom panels). (B) Representative reticulin-stained livers from wild-type and Iqgap2^{-/-} mice showing HCC. Note the discontinuous patterns of reticulin fibers in the HCC liver tissue. Scale bars correspond to 200 μm.

FIG. 11. Molecular analysis of HCC in Iqgap2^{-/-} mice. Immunohistochemical detection of IQGAP1, β-catenin, and E-cadherin in livers from 4-month-old wild-type or Iqgap2^{-/-} mice without HCC or 2-year-old Iqgap2^{-/-} mice with HCC. Note the loss of E-cadherin from cell membranes, along with the enhanced cytoplasmic expression and translocation of both IQGAP1 and β-catenin, occurring only with HCC. Scale bars correspond to 50 μm.

FIG. 12. (A) Immunoblot analysis was completed using an anti-β-catenin monoclonal antibody and liver lysates (10 μg/lane) from mice (2 years old) with HCC or age-matched controls; the arrow indicates the 92-kDa β-catenin band, with clear evidence for truncated β-catenin mutant forms in the case of HCC. (B) Immunoblot (10 μg of lysate/lane) demonstrating the presence of active (dephosphorylated) β-catenin mutant forms in HCC liver tissue. Results for livers from two 4-month-old wild-type (WT) and Iqgap2^{-/-} (−/−) mice are also shown. Cell lysate from the A431 cell line (Upstate Biotechnology/Millipore) was used as a positive control.

FIG. 13. (A) Immunoblot analysis (10 μg of lysate/lane) to assess E-cadherin, cyclin D1, and IQGAP1 expression in Iqgap2^{-/-} liver lobes without HCC or affected by HCC; paired normal (N) and tumor (T) samples were collected from the same animals. (B) Immunoblot densitometric analysis of E-cadherin, cyclin D1, and IQGAP1 expression was conducted using liver lysates (10 μg/lane) from lobes affected by HCC (n=4), those of 4- and 12-month-old Iqgap2^{-/-} mice without HCC (n=3), and those of 4-month-old wild-type mice (n=2). Data are presented as mean ratios ±standard deviations of the expression of individual proteins relative to that of proteins in the wild-type samples.

FIG. 14. (A) IQGAP1 expression was assessed by immunoblotting of liver lysates (10 μg/lane) from wild-type (WT) and Iqgap2^{-/-} (−/−) mice of various ages (6 weeks and 4, 12, and 24 months). Glyceraldehyde-3-phosphate dehydrogenase (GAPDH)-specific antibody served as a control for the loading of equal quantities. A representative immunoblot is shown. (B) Immunoblot densitometric analysis of hepatic IQGAP1 expression in wild-type and Iqgap2^{-/-} mice of various ages. Data are presented as means±SEM of the integrated optical densities of bands. n=3 for each age group.

FIG. 15. Molecular interactions involving IQGAPs, β-catenin, and E-cadherin. (A) Immunoprecipitations were completed using liver lysates (20 mg of total protein per sample) from 12-month-old wild-type (WT), Iqgap1^{-/-}, or Iqgap2^{-/-} mice and a protein G agarose-conjugated anti-β-catenin antibody (5 μg per sample). Complexes were fractionated by 4 to 15% gradient SDS-PAGE and immunodetected using antibodies specific to IQGAP1, IQGAP2, β-catenin, and E-cadherin. Results represent one complete set of experiments reproduced on two different occasions. (B) Immunoprecipitation (IP) experiments using COS1 lysates (20 mg of total protein per sample) transiently expressing enhanced green fluorescent protein (EGFP) as a control or human IQGAP1 or IQGAP2 (24 h post-transfection) were completed using protein G agarose-conjugated antibodies (5 μg per sample) directed against IQGAP1 or IQGAP2. Immunodetection was completed using the same antibodies. An immunoblot of corresponding lysates is shown to document protein expression. Note the high level of endogenous IQGAP1 expression in COST cells transfected with enhanced green fluorescent protein, with no evidence of IQGAP1-IQGAP2 complexes. Results represent one complete set of experiments reproduced on three different occasions.

FIG. 16. Immunoblots of liver lysates (30 μg/lane) from age-matched (4-month-old) Iqgap1^{-/-} Iqgap2^{-/-} (double-knockout [DKO]), Iqgap1^{-/-}Iqgap2^{+/-} double-heterozygote (HET/HET), and wild-type (WT) mice were obtained using either anti-IQGAP1 or anti-IQGAP2 antibodies. Antibody against actin was used to document the loading of equal amounts of protein.

FIG. 17. AST levels in sera from 18- to 24-month-old wild-type (n=12), Iqgap2^{-/-} (n=14), and Iqgap1^{-/-}Iqgap2^{-/-} (n=23) mice were determined; data are presented as the means±SEM, and p values are indicated.

FIG. 18. Aggregate Kaplan-Meier survival curves for wild-type, Iqgap2^{-/-}, and Iqgap1^{-/-}Iqgap2^{-/-} mice are displayed, with cohort numbers in parentheses; intergroup Mantel-Cox analyses demonstrated highly significant survival-rate differences between Iqgap2^{-/-} and either wild-type or Iqgap1^{-/-}Iqgap2^{-/-} mice (P=0.002), with no difference in survival rates between wild-type and Iqgap1^{-/-}Iqgap2^{-/-} mice.

FIG. 19. Liver weights as percentages of total body weights of wild-type mice (n=18) and Iqgap2^{-/-} (n=10) and Iqgap1^{-/-}Iqgap2^{-/-} (n=6) mice affected with HCC. The bar graph shows means±SEM, and P values are displayed.

FIG. 20. Immunohistochemical detection of IQGAP2 in normal human liver and liver with HCC. A representative set of images (n=3) is shown. Hepatocyte-specific antigen (HSA) was used as a positive control. Scale bars correspond to 50 μm.

FIG. 21. Either IQGAP1 or IQGAP2 expression in human hepatocellular carcinoma HepG2 cell line was downregulated using IQGAP1 or IQGAP2 specific siRNA. 48 hours later cell migratory ability was measured by a migration assay. Scrambled control represents an empty siRNA vector; WT—naïve cells without siRNA treatment.

### DETAILED DESCRIPTION OF THE INVENTION

The invention provides a non-human mammal, having a targeted gene disruption in an endogenous Iqgap2 gene. A targeted gene disruption in the endogenous Iqgap2 gene in mice can be achieved by homologous recombination in a mouse embryonic stem (ES) cell between the endogenous gene and a targeting construct. The targeting construct is designed to recombine with and alter a specific locus. Typically, the targeting construct contains a positive selection marker flanked by segments of the gene to be targeted. Usually the segments are from the same species as the gene to be targeted (e.g., mouse). However, the segments can be obtained from another species, provided they have sufficient sequence identity with the gene to be targeted to allow homologous recombination to occur. Typically, the construct also contains a negative selection marker positioned outside of the homologous segments in order to select against random integration events.

In addition, the construct can also contain a pair of site-specific recombination sites, such as loxP or frt, positioned within or at the ends of the segments designed to undergo homologous recombination with the endogenous gene. The presence of site-specific recombination sites allows for the removal of the targeting construct by expression of Cre recombinase (for LoxP sites) or the Flp recombinase (for frt sites). The recombinase can be expressed in the ES cells to remove a potion of the targeting cassette following homologous recombination. Alternatively, the site-specific recombination site can be introduced into a gene by homologous recombination. By breeding a mouse containing the site-specific recombination sites flanking all or a portion of a gene with a mouse that expresses the recombinase in a tissue specific fashion, the gene can be knocked out in a tissue-specific fashion in the resulting offspring.

The exogenous gene may be from the same species or from a different species than the animal host, or may be otherwise altered in its coding or non-coding sequence. The introduced gene may be a wild-type gene, naturally occurring polymorphism, or a genetically manipulated sequence, for example having deletions, substitutions or insertions in the coding or non-coding regions. The introduced sequence may encode an Iqgap2 polypeptide, or may utilize the Iqgap2 promoter linked to a reporter gene. Where the introduced gene is a coding sequence, it is usually linked to a promoter, which may be constitutive or inducible, and other regulatory sequences required for expression in the host animal. In addition, the promoter may be tissue specific.

In an embodiment of the invention, the targeting construct is introduced into ES cells, usually by electroporation, and undergoes homologous recombination with the endogenous gene, thereby incorporating the positive selection marker and parts of the flanking segments (and frt or loxP sites, if present) into the endogenous gene. ES cells that have undergone the desired recombination are selected by positive or negative selection. Positive selection selects for cells that have undergone the desired homologous recombination, and negative selection selects against cells that have undergone a random integration event. Following selection, ES cells are screened to determine whether the genome comprises a targeting construct by Southern blot analysis or PCR techniques.

Transformed ES cells are combined with blastocysts from a non-human animal for example a mouse. The ES cells are incorporated into the embryo and in some embryos form or contribute to the germline of the resulting chimeric animal. Chimeric animals can be bred with nontransgenic animals to generate heterozygous transgenic animals. Heterozygous animals can then be bred with each other to generate homozygous animals. In addition, non-human animals that are heterozygous or homozygous for a targeted gene disruption can be bred with a transgenic animal expressing the flp or cre recombinase. Expression of the recombinase results in excision of the portion of DNA between introduced frt or loxP sites, if present.

Targeted gene disruption can also be achieved for rodents such as rats and rabbits, ovines such as sheep, bovines such as cattle and buffalo, caprines such as goats, and porcines such as pigs. For animals other than mice, nuclear transfer technology is preferred for generating functionally inactivated genes. See Lai et al., Science 295: 1089-92 (2002). Various types of cells can be employed as donors for nuclei to be transferred into oocytes, including ES cells and fetal fibrocytes. Donor nuclei are obtained from cells cultured in vitro into which a targeted construct has been introduced and undergone homologous recombination with an endogenous gene, as described. Donor nuclei are introduced into oocytes by means of fusion, induced electrically, or by microinjection. Transplanted oocytes are subsequently cultured to develop into embryos which are subsequently implanted in the oviducts of pseudopregnant female animals, resulting in birth of transgenic offspring. Animals that are heterozygous for the targeted gene disruption can be bred with each other to generate animals that are homozygous for the targeted gene disruption.

In addition to a heterozygous or homozygous targeted disruption in the Iqgap2 gene, non-human mammals of the present invention may also have a targeted disruption in one or more other genes. For example, mice that are heterozygous for a targeted disruption in the Iqgap2 gene can be bred with mice with a targeted disruption in the Iqgap1 gene. Offspring that are double heterzygotes can be bred to create mice that are homozygous for targeted disruptions at both the Iqgap1 and Iqgap2 genes.

The non-human mammals of the invention can be either heterozygous or homozygous for a targeted gene disruption. Initial screening to determine whether a genome comprises a targeting construct can be accomplished by Southern blot analysis or PCR techniques. Further, endogenous Iqgap2 mRNA expression levels in tissues from a non-human mammal can be assessed using techniques that include, but are not limited to, Northern blot analysis of tissue samples obtained from the mammal, in situ hybridization analysis, and RT-PCR.

The targeted gene disruption in an endogenous gene creates a null allele that lacks (or substantially lacks) that gene's normal function. If the non-human mammal is homozygous for the null allele (i.e., a knock-out) the result is the complete absence of the gene product at the molecular level, or the expression of a non-functional gene product. This result may be achieved by a variety of mechanisms, including introduction of a targeted gene disruption of the coding sequence, e.g., by insertion of one or more stop codons, insertion of a DNA fragment, deletion of coding sequence, substitution of stop codons for coding sequence, etc. Targeted gene disruptions also include conditional knock-outs, for example where alteration of the target gene occurs upon exposure of the animal to a substance that promotes target gene alteration, introduction of an enzyme that promotes recombination at the target gene site (e.g. Cre in the Cre-lox system or Flp in the frt-Flp system), or other method for directing the target gene alteration postnatally.

A functional knock-down or knock-out may be achieved by the introduction of an anti-sense construct that blocks expression of the endogenous genes (for example, see Li and Cohen (1996) Cell 85:319-329) or by introduction of a construct that expresses a siRNA that inhibits the expression of the target gene. These RNA expression constructs may contain a tissue-specific promoter to direct the expression of the RNA, and the subsequent inhibition of the target gene expression, to a specific tissue or tissues.

In addition to exhibiting a particular genotype, a transgenic non-human animal of the invention also can exhibit one or more useful phenotypes. For example, a transgenic non-human mammal that is homozygous for a targeted disruption in the endogenous Iqgap2 gene can develop an age-dependent increase in hepatocellular carcinoma. In addition, a transgenic non-human mammal that is homozygous for a targeted disruption in the endogenous Iqgap2 gene can develop an age-dependent increase in apoptosis of hepatocytes. A transgenic non-human animal of the present invention that contains a transgene that expresses an RNA (antisense, or siRNA) that inhibits Iqgap2 expression can also develop an age-dependent increase in hepatocellular carcinoma and/or an age-dependent increase in apoptosis of hepatocytes.

Cell lines of the present invention can be derived from the non-human mammals containing a targeted gene disruption in an endogenous Iqgap2 gene. Sources of cell lines include cells from various tissues containing a targeted gene disruption in an endogenous Iqgap2 gene including, but not limited to, hepatocytes, fibroblasts, myocytes, adipocytes, lymphocytes, megacaryocytes, and platelets.

Cell lines of the present invention may also have expression of Iqgap2 reduced or eliminated by introduction or expression of an antisense RNA or a small interfering RNA (siRNA). Such cells include, but are not limited to, human and non-human stem cells, adult stem cells, cancer stem cells, hepatocytes, fibroblasts, myocytes, adipocytes, lymphocytes, megacaryocytes, and platelets, as well as their precursors and progenitors. Transient reduction of Iqgap2 expression can be achieved by introducing an siRNA into a cell line by standard transfection techniques. In addition, antisense RNA, or siRNA can be expressed from an expression construct that is introduced into the cells by standard transfection techniques or through the use of adenoviral systems. Antisense RNA, or siRNA may be expressed from constructs that incorporate into the genome of the cells using e.g., retroviral or lentiviral constructs. Expression vectors for expression of siRNAs are commercially available from vendors such as Clontech, Invitrogen, Millipore, Gene Therapy Systems, Ambion and Stratagene.

Appropriate siRNAs are designed using methods known in the art (see e.g., Brummelkamp, T. R. et al. (2002) A system for stable expression of short interfering RNAs in mammalian cells. Science 19, 550-553; Ui-Tei, K. et al. (2004) Guidelines for the selection of highly effective siRNA sequences for mammalian and chick RNA interference. Nucleic Acids Res. 32, 936-948; Hohjoh H. (2004) Enhancement of RNAi activity by improved siRNA duplexes. FEBS Lett. 557, 193-8; and Yuan, B., et al. siRNA Selection Server: an automated siRNA oligonucleotide prediction server. (2004) Nucleic Acids Res. 32, W130-134).

Cell lines with a targeted gene disruption in the Iqgap2 gene and cell lines that contains an antisense, or siRNA that inhibits Iqgap2 expression can also display a particular phenotype. For example, observable phenotypes of Iqgap2^{-/-} cell lines useful for screening include: cell viability (by MTT assay), cell morphology, proliferation, migration, invasiveness (Matrigel assay), cell adhesion, mitochondrial structure, and apoptosis (TUNEL assay, for instance). In addition, phenotypes of Iqgap2 targeted gene disruption can be monitored by E-cadherin and beta-catenin translocation within a cell and levels of cyclin D1 expression in nuclei by immunohistochemistry and immunoblotting of various cellular fractions.

The invention also makes evident the functional involvement of IQGAP1 in HCC development. For example, while survival and HCC incidence barely differ between wild type mice and mice carrying an IQGAP2 disruption together with an IQGAP1 disruption (i.e., Iqgap1^{-/-}/Iqgap2^{-/-} mice) at intermediate ages (e.g., 12-18 months), survival is significantly reduced and HCC incidence increased where IQGAP1 function is present (i.e., in Iqgap2^{-/-} mice). (See, e.g., FIG. 18). Non-human mammals of the present invention may contain a transgene that encodes an Iqgap1 polypeptide. The expression of Iqgap1 from the transgene can be under the control of a tissue-specific promoter or an inducible promoter. The transgene construct can be introduced into ES cells by standard techniques, which are then injected into blastocycts as described above. In addition, the transgene construct can be introduced by pronuclear injection into an egg cell following fertilization. For pronuclear injection, a fine needle is used to inject the transgene construct into the pronucleus, which is derived from the sperm.

### Drug Screening Assays

The present invention features a method of identifying a compound as a therapeutic agent for the treatment of hepatocellular carcinoma, by contacting the compound with a test cell that underexpresses Iqgap2, wherein Iqgap1 is overexpressed in response to Iqgap2 underexpression, and identifying the compound as a therapeutic agent for treatment of hepatocellular carcinoma if IQGAP1 activity is reduced in response to the compound. The test cell may under-express Iqgap2 due to a targeted gene disruption in the Iqgap2 gene or due to the expression of an antisense RNA, or siRNA that is designed to reduce the expression of Iqgap2. Test cells include, but are not limited to, hepatocytes, fibroblasts, embryonic fibroblasts, platelets, and other cell types described herein, as well as their precursors and progenitors and cell lines derived therefrom. IQGAP1 activity may be monitored by northern blotting, immunoblotting, immunohistochemistry, or quantitative PCR. In addition, the IQGAP1 activity may be evaluated by observing the level of Iqgap1 expression using an Iqgap1 reporter gene construct. The reporter gene construct may comprise the Iqgap1 regulatory sequences (promoter, enhancers) linked to a reporter gene such as luciferase.

The invention also features a method of identifying a compound as a therapeutic agent for the treatment of hepatocellular carcinoma, by contacting the compound with a test cell that over-expresses Iqgap1, and identifying the compound as a therapeutic agent for treatment of hepatocellular carcinoma if IQGAP1 activity is reduced in response to the compound. The test cell may be, for example, a hepatocyte or a fibroblast. IQGAP1 activity may be monitored by northern blotting, immunoblotting or quantitative PCR. In addition, the IQGAP1 activity may be evaluated by observing the level of Iqgap1 expression using an Iqgap1 reporter gene construct. For example, the reporter gene construct may comprise a the Iqgap1 regulatory sequences (promoter, enhancers) linked to a reporter gene such as luciferase.

In an embodiment of the invention, a cell or cell line of the invention, in which IQGAP2 is underexpressed or not expressed at all is used to identify compounds that modulate phenotypes associated with IQGAP1 function. In one embodiment, IQGAP1 is overexpressed in the cell as a consequence of the IQGAP2 deficiency. In another embodiment, the cell is engineered to overexpress IQGAP1, for example by transient or stable transfection of an endogenous IQGAP1 gene construct. In another embodiment, the IQGAP2 deficient cell line is cultured under conditions that activate IQGAP1 function. Useful phenotypes include, but are not limited to, cell viability, cell morphology, proliferation, migration, invasiveness, cell adhesion, cytoskeletal structure, mitochondrial structure, and apoptosis. Other non-limiting phenotypes include regulation of EGF-stimulated MEK and ERK activity and modulation of B-Raf activity, translocation of E-cadherin or beta-catenin, and cyclin D1 expression. In an embodiment of the invention, to identify a modulator that acts directly on (i.e., binds to) IQGAP1 or a complex comprising IQGAP1, a phenotype is chosen that is correlated to the level of IQGAP1 activity (i.e., the amount of IQGAP1 and its specific activity) in the IQGAP2 deficient cell when IQGAP1 is overexpressed. Activators or inhibitors of IQGAP1 activity are identified by their activation or inhibition of the observed phenotype.

The invention also features a method of identifying a compound as a therapeutic agent for the treatment of hepatocellular carcinoma, by providing a non-human mammal, which comprises a targeted gene disruption in an endogenous Iqgap2 gene and exhibits an age-dependent increase in apoptosis of hepatocytes, administering the compound to the non-human mammal, and identifying the compound as a therapeutic agent if it reduces the age-dependent increase in apoptosis.

The invention features a method of identifying a compound as a therapeutic agent for the treatment of hepatocellular carcinoma, by providing a non-human mammal which comprises a targeted gene disruption in an endogenous Iqgap2 gene and exhibits an age-dependent increase in hepatocellular carcinoma; administering the compound to the non-human mammal, and identifying the compound as a therapeutic agent if it reduces the age-dependent increase in hepatocellular carcinoma.

Therapeutic agents for the treatment of hepatocellular carcinoma include, but are not limited to, small molecules, antibodies, polynucleotides, and polypeptides.

### Treating Hepatocellular Carcinoma

The present invention provides a method of treating or preventing hepatocellular carcinoma by administering an agent that decreases Iqgap1 function in hepatocytes. One way to decrease Iqgap1 function is through the use of small interfering RNAs (siRNAs). IQGAP1-specific oligonucleotides (19-21 nucleotides) can be synthesized and converted into double-stranded RNA molecules that can target IQGAP1 mRNA in the cell with complementary sequence for degradation via a process known as RNA interference thus reducing expression of IQGAP1 protein. IQGAP1-siRNA can be delivered to the organism by methods including lyposomes, nanoplexes or Ad 5 adenovirus vectors. Such interfering RNAs can be partially or completely composed of nucleotide analogs such as thionucleotides or locked nucleic acids. There are various possible routes of administration including by intravenous, intraperitoneal, intra-tumor injections or injection into the portal vein.

Another way to decrease Iqgap1 function is through the use synthetic blocking peptides directed against specific IQGAP1 domains. IQGAP1 domain structure includes: actin-binding calponin homology (CH) domain, SH3-mimicking domain (WW), calmodulin-binding domains (IQ), and GTPase-binding domain (GBD). The WW domain has been shown to bind extracellular signal-regulated kinases ERK1 and ERK2; IQ domain binds MEK1 and MEK2 kinases and calmodulin; GBD binds specifically only Rho GTPases Rac1 and cdc42; and the C-terminal domain is responsible for binding E-cadherin, beta-catenin, CLIP-170, APC, and the exocyst complex components Ses8 and Sec3, involved in matrix degradation and cell invasion.

The present invention provides a method of treating or preventing hepatocellular carcinoma by administering an agent that enhances Iqgap2 function in hepatocytes. One way to enhance Iqgap2 function is through hepatic gene transfer. IQGAP2 protein can be supplemented by delivering an IQGAP2 gene to the liver using various vectors with liver-specific promoters. Both non-viral (recombinant plasmids) and viral vectors (adenoviruses, adeno/adeno-associated (Ad/AAV) hybrid virus and retroviruses) can be used. Liver-specific promoters include, but are not limited to, promoters derived from albumin, α₁-antitrypsin (AAT), phosphoenolpyruvate carboxykinase (PEPCK) and fatty acid binding protein (FABP) genes. A full-length cDNA encoding IQGAP2 gene will be cloned into a vector under control of chosen liver-specific promoter and the resulting construct will be delivered to the organism by one of the following: intravenous, portal vein, intraperitoneal or intra-tumor injections. Non-viral (plasmid-based) constructs can be delivered by liposomes or nanosomes (nanoplexes).

IQGAP2 expression may also be increased using methylation inhibitors. Two groups of inhibitors of DNA methylation can be used: nucleoside inhibitors (5-azacytidine, 5-aza-2′-deoxy-cytidine, zebularine, 5-fluoro-2′-deoxycytidine) and non-nucleoside inhibitors (procainamide, hydralazine). Possible routes of administration include orally, intravenously or intra-tumor injection.

The techniques described herein for the generation of targeting constructs, PCR, Southern blotting, ES cell culture and manipulation, and breeding of transgenic mice are found in Sambrook et al., 1989, Molecular Cloning, A Laboratory Manual, second edition, Cold Spring Harbor Press, Plainview, N.Y. A. L. Joyner, 2000; Gene Targeting: A Practical Approach, Oxford University Press, New York. Nagy et al., 2003; and Manipulating the Mouse Embryo: A Laboratory Manual, Cold Spring Harbor Press, Plainview, N.Y. All references cited herein are incorporated in their entirety.

### EXAMPLES

### Example 1

### Generation of Iqgap^{-/-} Mice

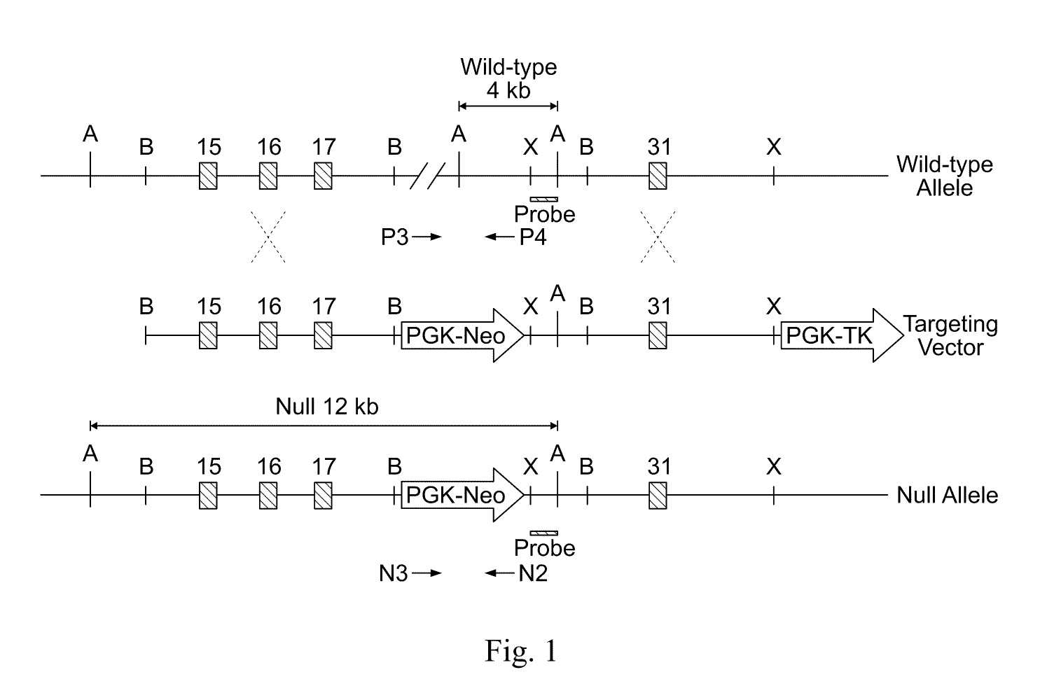

An 150-kb bacterial artificial chromosome clone derived from murine chromosome 13 and encompassing exons 7 to 34 of the Iqgap2 gene was isolated as previously described (Cupit, L. D., et al. 2004. Genome 15: 618-629). The targeting vector was constructed by the insertion of an 8.5-kb genomic fragment containing Iqgap2 exons 15 to 17 into the BamHI site of plasmid pMJK-KO, upstream from the phosphoglycerate kinase promoter-neomycin (PGK-Neo) reporter cassette. An 8-kb Iqgap2 genomic fragment encompassing exon 31 was cloned into the XhoI site downstream of the PGK-Neo cassette. The targeting vector also contained a thymidine kinase gene (PGK-tk) as a second selection marker (see FIG. 1). The linearized targeting vector was introduced into 129J1 embryonic stem (ES) cells by electroporation, and the Iqgap2 null allele was generated by homologous recombination using dual selection (Mansour, S. L., et al. 1988. Nature 336:348-352). To inactivate the Iqgap2 gene, a 36,241-bp genomic fragment spanning exons 18 to 30 (corresponding to amino acids Ala705 to Lys1306, encompassing a portion of the IQ2 [Gln696 to Gly725] motif, the entire IQ3 [Asn726 to Asp755] and IQ4 [His756 to Pro785] motifs, and the GRD [Gln891 to Val1189]) was replaced with a PGK-neomycin resistance cassette via site-specific homologous recombination (FIG. 1).

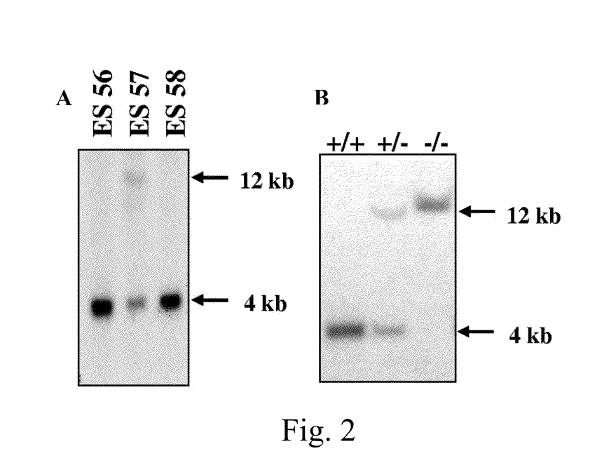

Genomic DNA from individual ES clones was screened by Southern analysis using ApaI-digested DNA and a ³²P-radiolabeled, PCR-generated 500-bp fragment as a probe (forward, 5′-GCTGGCAGTGGGGAGCACAGTGCA-3′, SEQ ID NO:1; reverse, 5′-AGGGCAGGAAAGGCAGCAGCACTT-3′, SEQ ID NO:2) (FIG. 2A). ES clone no. 57 was used for microinjections into C57BL/6 blastocysts to generate chimeric mice (Mansour, S. L. et al.) and bred into 129J1 and C57BL/6 backgrounds for at least eight generations prior to further studies. All functional studies were completed using 129J1 mice. F1 progeny of the chimeric mice were initially genotyped by Southern blot analysis (as outlined above) using tail DNA (FIG. 2B).

Genotyping of subsequent progeny was completed by PCR. Oligonucleotide pair P3/P4 (P3: 5′-AAGGCATGATTCATTCACCTGAGA-3′, SEQ ID NO:3; P4: 5′-AGGGCAGGAAAGG CAGCAGCACTT-3′, SEQ ID NO:4) was used for the detection of the wild-type allele, and N2/N3 (N2: 5′-GTCAAGAAGGCGATAGAAGG-3′, SEQ ID NO:5; N3: 5′-TTGAACAAGATGGATTGCACGCA-3′, SEQ ID NO:6) was used to detect the null allele. The 35-cycle PCR included a denaturation step at 94° C. for 1 min and 15 s, a 2 minute annealing step at 55° C., and a 3 minute primer extension step at 72° C. Mice with targeted disruption of the Iqgap1 gene were maintained on a 129J1 background. Iqgap1^{-/-} Iqgap2^{-/-} mice were generated by pairwise interbreeding of Iqgap1^{-/-} and Iqgap2^{-/-} mice with the 129J1 background. Offspring were born with predicted Mendelian frequencies, establishing that the Iqgap1^{-/-}Iqgap2^{-/-} genotype did not result in embryonic lethality.

Interbreeding of heterozygous (Iqgap2^{-/+}) founder mice on both 129J1 and C57BL/6 backgrounds demonstrated that progeny were born at normal Mendelian ratios, eliminating the possibility that Iqgap2 expression is required for normal embryonic development. Iqgap2^{-/-} mice were fertile and clinically normal at up to 12 months of age.

### Example 2

### Characterization of Iqgap^{-/-} Mice

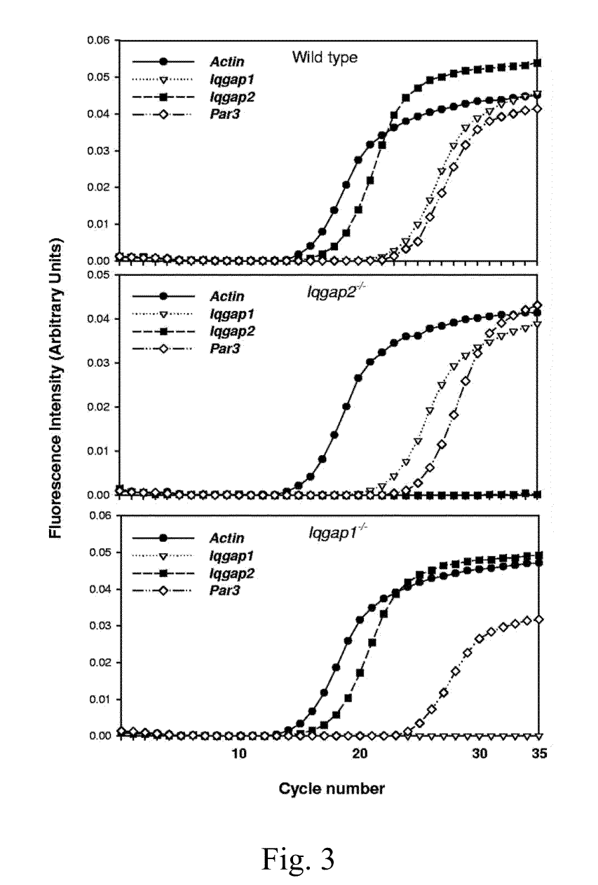

To exclude the possibility that Iqgap2 loss caused the reciprocal induction of the homologous Iqgap1 gene, qRT-PCR was completed using hepatocytes from Iqgap1^{-/-} and Iqgap2^{-/-} mice (FIG. 3). In parallel, transcript expression of the murine protease-activated receptor 3 (Par3) gene was assessed to exclude inadvertent disruption. Murine Par3 is known to be encompassed within Iqgap2 intron 13, and unaltered expression established that the thrombin-PAR3 receptor system was uninvolved in subsequent phenotypic defects. Transcript analysis confirmed that Iqgap2 was the predominant isoform expressed in murine livers, with no clear change in Iqgap1 gene expression.

Quantitative reverse transcription-PCR (qRT-PCR) was performed using fluorescence-based real-time PCR technology (Gnatenko, D. V. et al. 2003. Blood 101:2285-2293); in brief, 5 μg of total RNA from murine livers was used for first-strand cDNA synthesis with random hexamer primers (Invitrogen, Carlsbad, Calif.). Total RNA in 7 μl of H₂O was mixed with 5 μl of hexamer primers (100 ng/μl), and the mixture was heated to 85° C. for 3 min and placed on ice. The reaction mix was prepared separately according to the Invitrogen protocol and contained 4 μl of 5× first-strand buffer, 2 μl of 0.1 M dithiothreitol, and 1 μl of a mix of deoxynucleoside triphosphates (15 mM each). RNA with primers was then added to the reaction mix, the mixture was incubated at 42° C. for 3 min, and the reverse transcription was started by the addition of 1 μl of SuperScript II reverse transcriptase (200 U/μl; Invitrogen [catalog no. 18064]). The reverse transcription reaction was carried out at 42° C. for 2 h and stopped by incubation of the reaction mixture at 85° C. for 10 min. The following PCR step was performed in a 96-well plate using an Opticon-I PCR machine (Bio-Rad). The reverse transcription reaction mixture was diluted up to 1:15 and equally divided among primer pairs for each target gene (Table 1).

TABLE 1
Oligonucleotide primers for qRT-PCR

| | | SEQ | | SEQ |
|---|---|---|---|---|
| | | ID | | ID |
| Gene | Forward primer | NO: | Reverse primer | NO: |
| Iqgap1 | 5′-TGCAGCTGACACTTT | 7 | 5′-TTCCCTTGGATTTCA | 8 |
| | TACGG-3′ | | GCTTG-3′ | |
| Iqgap2 | 5′-TCAAGATTGGACTGC | 9 | 5′-AGGTTTGGTCTGGAG | 10 |
| | TGGTG-3′ | | GAGGT-3′ | |
| Par3 | 5′-TCAATGGCAACAACT | 11 | 5′-ATGACCCACACTATG | 12 |
| | GGGTA-3′ | | CCACA-3′ | |
| β-Actin | 5′-TACCACAGGCATTGT | 13 | 5′-TTTGATGTCACGCAC | 14 |
| gene | GATGG-3′ | | GATTT-3′ | |
| 16SrRNA | 5′- | 15 | 5′-CCCCAACCGAAATTT | 16 |
| | GGGACTAGCATGAAC | | CAAAC-3′ | |
| | GGCTA-3′ | | | |
| COX | 5′-CTGAGCGGGAATAGT | 17 | 5′-TGGGGCTCCGATTAT | 18 |
| (cytoc- | GGGTA-3′ | | TAGTG-3′ | |
| hrome c | | | | |
| oxidase | | | | |
| subunit | | | | |
| 1 | | | | |
| Cyto- | 5′-ACGTCCTTCCATGAG | 19 | 5′-GAGGTGAACGATTGC | 20 |
| chrome b | GACAA-3′ | | TAGGG-3′ | |
| gene | | | | |

Oligonucleotide primer pairs for each target gene were generated using Primer3 software (http://frodo.wi.mit.edu/cgi-bin/primer3/primer3_www.cgi) and were designed to amplify PCR products of 200±1 by at the same annealing temperatures. The plate was heated at 95° C. for 15 min and subjected to a 40-cycle PCR (94° C. for 30 s, 55° C. for 30 s, and 72° C. for 1 min and fluorescence reading for 10 seconds). Melting curves were measured for every well of the plate at temperatures from 65 to 95° C., with a reading interval of 0.2° C. and 5 s of holding between the readings. mRNA levels were quantified by monitoring the real-time fluorimetric intensity of SYBR green I (Invitrogen). Known amounts of DNA were used as standards to generate a calibration curve covering a range from 1 to 10⁻⁶ ng of a target per well. The relative mRNA abundance was determined from triplicate assays performed in parallel for each primer pair and calculated using the comparative threshold cycle number (Heid, C. A., et al. 1996. Genome Res. 6:986-994), and results were normalized to the amount for cellular β-actin mRNA, all as previously described (Gnatenko, D. V., et al. 2005. Thromb. Haemost. 94:412-421).

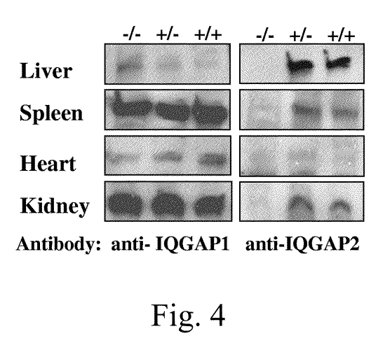

Immunoblot analysis using tissues known to express Iqgap2 (14) demonstrated a clear loss of protein, with the exception of a modest increase in hepatic IQGAP1 content (FIG. 4). Thus, comparable to the Iqgap1^{-/-} mouse, the Iqgap2^{-/-} genotype did not result in intrauterine or immediate postnatal death, nor was there any evidence that compensatory induction of Iqgap1 masked the lack of an essential Iqgap2 function in adult tissues.

Protein lysates from various organs were prepared as previously described (Schmidt, V. A., et al. 2003. IQGAP2 functions as a GTP-dependent effector protein in thrombin-induced platelet cytoskeletal reorganization. Blood 101:3021-3028.). Lysates were centrifuged at 600×g for 10 min at 4° C., and protein supernatants were quantified by a bicinchoninic acid assay prior to sodium dodecyl sulfate-polyacrylamide gel electrophoresis (SDS-PAGE) using 4 to 15% gradient gels (Bio-Rad Laboratories, Hercules, Calif.). Immunoblot analysis was completed as previously described (Schmidt, V. A., et al.). Primary antibodies were mouse monoclonal antibodies used at a 1:1000 dilution for IQGAP1 (BD Biosciences) and for IQGAP2 (Upstate Biotechnology/Millipore).

### Example 3

### Murine Phenotypic and Electron Microscopy Analyses

Freshly isolated mouse organs were immediately snap-frozen in liquid nitrogen or paraffin embedded by submersion in 10% neutral buffered formalin for 16 h at 4° C. prior to being sectioned and stained using hematoxylin and eosin.

The presence of HCC was established by the examination of the gross morphological features and a microscopic review of at least two sections per liver; for all specimens, microscopy was carried out in a blinded fashion and complete concordance was observed between the results of the examination of gross morphology and microscopic HCC detection. Histologic confirmation of HCC was established on paraffin sections using Gordon and Sweet's silver reticulin stain.

Biochemical determinations (for hepatic transferases, bilirubin, and alkaline phosphatase) were completed using sera isolated from retroorbital blood samples and quantified using a MODULAR PP serum work area analyzer (Roche Diagnostics, Indianapolis, Ind.). Automated blood counts were obtained on a Coulter counter specifically gated to detect murine blood cells, and blood smears were subjected to Wright-Giemsa stain.

### Example 4

### Iqgap2^{-/-} Mice Display Age-Dependent Hepatocellular Apoptosis

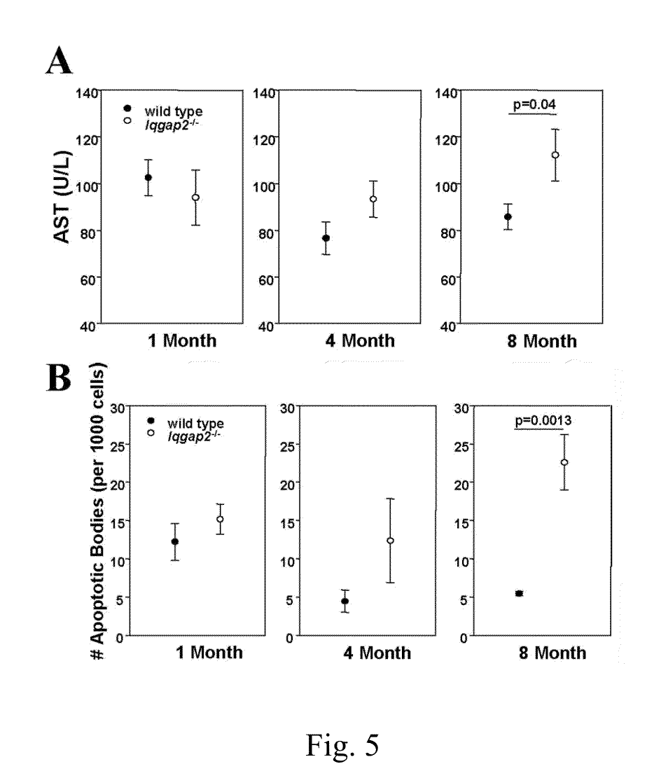

The initial survey of organs from 6-week-old Iqgap2^{-/-} mice demonstrated no histological defects in any of the tissues studied; similarly, animals were fertile and maintained normal body weights. More-detailed hematological and biochemical studies of mice from distinct age groups (1, 4, and 8 months old) revealed normal hemograms, although Iqgap2^{-/-} mice demonstrated an age-dependent increase in levels of distinct hepatocyte-derived aminotransferases in sera. Total bilirubin levels remained normal in mice up to 8 months of age, and enzymatic abnormality at this age was restricted to elevations of aspartate aminotransferase (AST) (FIG. 5A). A nonstatistically significant rise in AST was evident at 4 months, with clear differences in 8-month-old Iqgap2^{-/-} mice (P=0.04) compared to wild-type littermates. The AST was most likely hepatic (nonmuscular) in origin, since corresponding serum creatine phosphokinase levels remained normal and values for the two groups were statistically indistinguishable (53.6±6.3 U/liter in wild-type mice versus 72.4±20.59 U/liter in Iqgap2^{-/-} mice). Similarly, the extensive organ survey failed to identify histologic defects in any nonhepatic sources.

TUNEL staining using livers from wild-type and Iqgap2^{-/-} littermates specifically demonstrated no differences in the numbers of apoptotic cells in 1-month-old mice; however, gradual and progressive evidence for spontaneous apoptosis in 4- and 8-month-old Iqgap2^{-/-} mice was observed (FIG. 5B). Enhanced hepatocellular loss was evident (but not statistically significant) by 4 months, and that in 8-month-old mice was highly significant (P=0.0013). Thus, the biochemical evidence for liver damage paralleled that for an associated age-dependent accumulation of apoptotic hepatocytes.

The quantification of apoptotic hepatocytes was completed using terminal deoxynucleotidyltransferase-mediated dUTP-biotin nick end labeling (TUNEL) staining (Li, et al. 1995. Cytometry 20:172-180). Briefly, liver sections were deparaffinized in xylene, rehydrated in decreasing alcohol concentrations (from 100 to 0%), and digested with 20 μg/ml of proteinase K for 10 min at 25° C., and the digestion was subsequently quenched by incubation with 3% hydrogen peroxide in phosphate-buffered saline for 10 min at 25° C. Washed sections were then incubated for 60 min at 37° C. with terminal deoxynucleotidyltransferase enzyme for dUTP-biotin nick end labeling (Chemicon, Temecula, Calif.), after which they were incubated for 1 h at 25° C. with the digoxigenin-conjugated anti-dUTP antibody. Detection was completed using 3,3′-diaminobenzidine as the substrate. Sections were counterstained with 0.5% methyl green. Apoptotic cells (among 1,000 cells per section) were scored and quantified in a blinded fashion by two investigators, and the results were expressed as the mean±the standard error of the mean (SEM) for each age group studied.

### Example 5

### Structural Mitochondrial Abnormalities in Iqgap2^{-/-} Hepatocytes

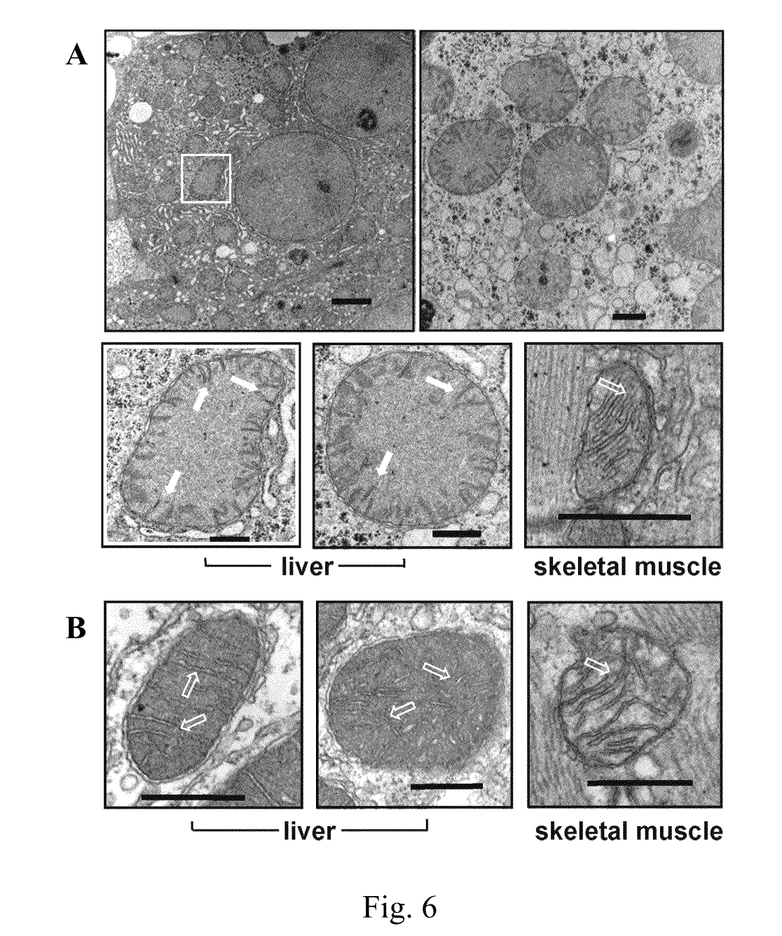

The age-related onset of the apoptotic phenotype evident in Iqgap2^{-/-} hepatocytes is characteristic of many mitochondrial diseases and is postulated to result from the senescent decline in oxidative phosphorylation seen in postmitotic tissues such as liver. An ultrastructural review of hepatocytes from Iqgap2^{-/-} mice (ages 1, 4, 8, and 12 months; n=3 per age group) displayed distinct age-dependent abnormalities restricted to mitochondria (FIGS. 6A and 6B). These defects were not present in any age-matched wild-type mice examined (n=8), were more pronounced with age, and were restricted to the liver (to the exclusion of skeletal muscle).

For electron microscopy, freshly isolated mouse liver and skeletal muscle samples were fixed by immersion in a solution of 2% paraformaldehyde and 2.5% electron microscopy-grade glutaraldehyde in 0.1 M sodium cacodylate buffer (pH 7.4). After fixation, samples were placed in 2% osmium tetroxide in 0.1 M sodium cacodylate buffer (pH 7.4), dehydrated in a graded series of ethyl alcohol preparations, and embedded in Durcupan resin (Electron Microscopy Sciences, Fort Washington, Pa.). Ultrathin sections of 80 nm were generated with a Reichert-Jung UltracutE ultramicrotome (Leica Microsystems, Inc., Bunnockburn, Ill.) and placed on Formvarcoated slot copper grids. Sections were then counterstained with uranyl acetate and lead citrate and viewed with a Tecnail2 BioTwinG2 electron microscope (FEI Company, Hillsboro, Oreg.). Digital images were acquired using an AMT XR-60 charge-coupled device digital camera system (Advanced Microscopy Techniques Co., Danvers, Mass.). All microscopic and ultramicroscopic analyses were performed blindly to the genotype.

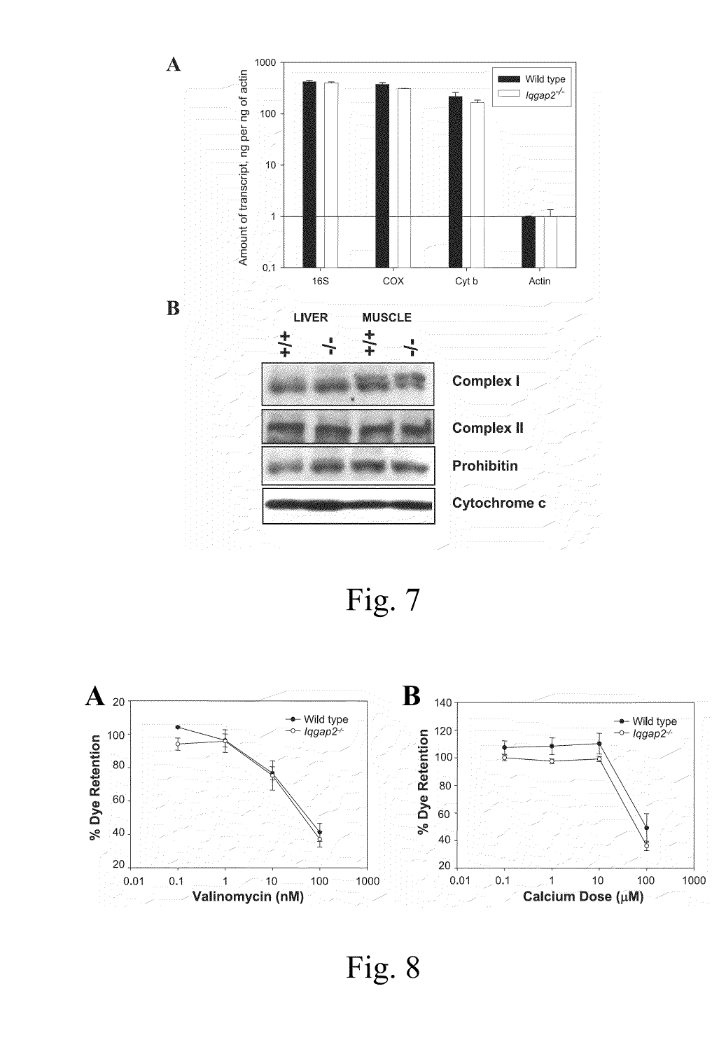

Furthermore, there was no evidence for associated abnormalities of mitochondrial number, as assessed by qRT-PCR analysis of mitochondrial RNA transcripts, or immunoblot analysis of key mitochondrial proteins (FIGS. 7A and B). qRT-PCR analysis was performed as described above using total liver RNA from 12-month-old Iqgap2^{-/-} mice or age-matched wild-type controls and primer pairs for the 16S subunit rRNA (16S), cytochrome c oxidase subunit 1 (COX), and cytochrome b (Cyt b) with the primer pairs listed in Table 1. Immunoblot analysis of key mitochondrial proteins was performed as described above but with liver and skeletal muscle lysates from wild-type and Iqgap2^{-/-} mice using antibodies specific to complex I, complex II, prohibitin, and cytochrome c.

In addition, the functional competence of the mitochondrial permeability transition pore (mtPTP; the complex regulating cytochrome c release as a primary apoptotic signal) was found to be normal, as assessed by comparable dose response sensitivities to calcium (the fundamental mtPTP activator) and valinomycin (a potassium-specific inducer of the mtPTP) (FIGS. 8A and B). Mitochondria were isolated from individual livers after isotonic homogenization in an ice-cold buffer containing 0.25 M sucrose, 10 mM HEPES buffer, pH 7.4, and 1 mM EGTA. The mitochondrial fraction was obtained by differential centrifugation at 4° C. (von Ahsen, et al. 2000. J. Cell Biol. 150:1027-1036), and the protein content was measured by a bicinchoninic acid assay (Pierce, Rockford, Ill.). The integrity and functional capacity of mitochondria were assessed by tetramethylrhodamine ethyl ester loading (50 mM for 10 min at 25° C.) and FACScan analysis using logarithmic-gain settings for light scattering and fluorescence (Becton Dickenson, Mountain View, Calif.). For all studies, 10,000 gated events were acquired.

Collectively, these data suggested that IQGAP2 deficiency caused hepatocellular apoptosis through a mitochondrial pathway unrelated to a concomitant intrinsic defect of mitochondrial function.

### Example 6

### Iqgap2^{-/-} Mice Develop Age-Related HCC

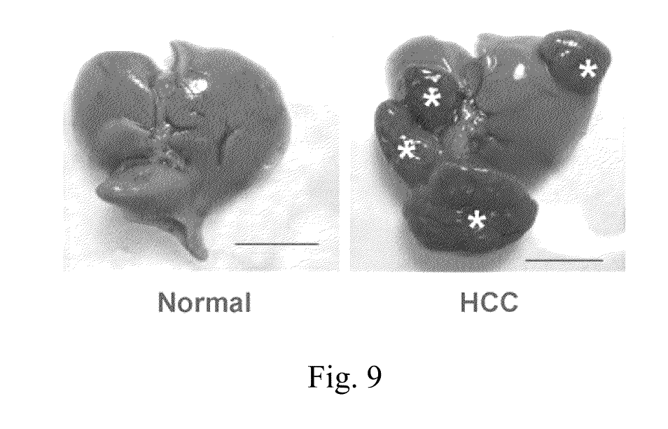

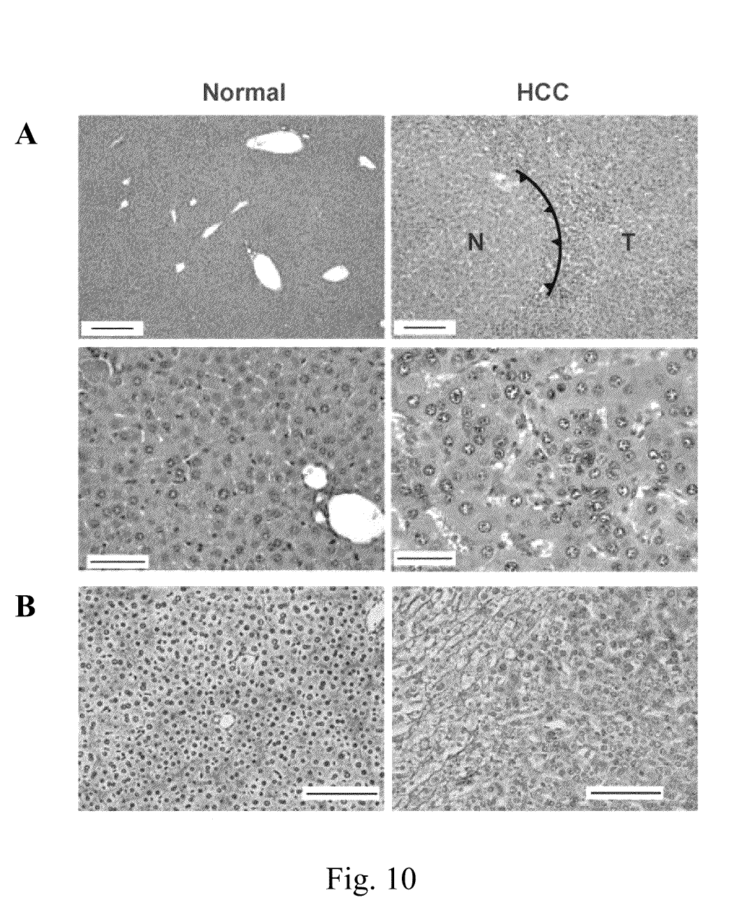

Since apoptosis is a hallmark of many hepatocellular disorders, mice were monitored for up to 2 years after birth. Detailed necroscopy of 18- to 24-month-old mice demonstrated a high incidence of HCC in Iqgap2^{-/-} mice (18 of 21; 86%) compared to that in wildtype controls (0 of 15) (P<0.0001) (see below). Both sexes were equally affected. In all situations, there was complete concordance between the presence of grossly abnormal livers and microscopic features of HCC (i.e., there were no instances of microscopic HCC in visibly normal livers) (FIG. 9); histologically, the neoplasms displayed well-differentiated features with trabecular and pseudoglandular patterns (FIG. 10A). More detailed histological analysis using reticulin staining confirmed the presence of distorted architecture of the hepatic cords in Iqgap2^{-/-} liver tumors, findings typically identified in HCC (FIG. 10B). Except for the occasional presence of uterine hemangiomas, no other tumors or abnormalities in Iqgap2^{-/-} mice were detected. Similarly, no steatosis or other hepatic abnormalities were evident.

To perform this microscopic analysis, freshly isolated mouse organs were immediately snap-frozen in liquid nitrogen or paraffin embedded by submersion in 10% neutral buffered formalin for 16 h at 4° C. prior to being sectioned and stained using hematoxylin and eosin. The presence of HCC was established by the examination of the gross morphological features and a microscopic review of at least two sections per liver; for all specimens, microscopy was carried out in a blinded fashion and complete concordance was observed between the results of the examination of gross morphology and microscopic HCC detection. Histologic confirmation of HCC was established on paraffin sections using Gordon and Sweet's silver reticulin stain.

### Example 7

### The Development of HCC in Iqgap2^{-/-} Mice is Associated with the Reciprocal Induction of IQGAP1 Expression and the Activation of the Wnt/β-Catenin Pathway

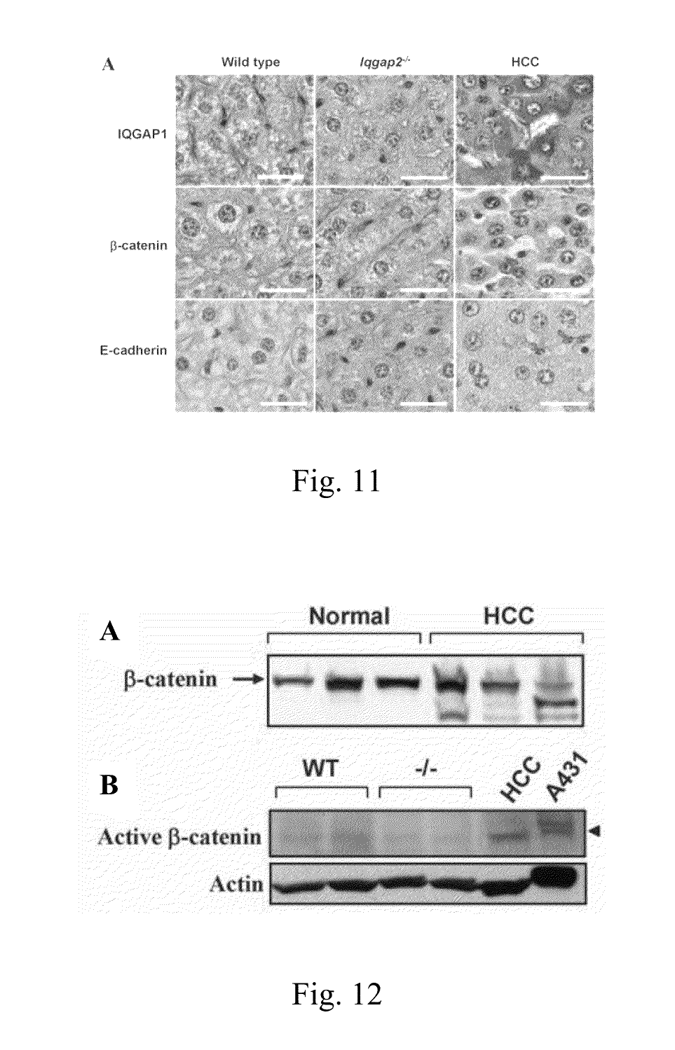

Since the Wnt/β-catenin pathway is frequently involved in the development of human HCC, hepatocellular expression patterns for β-catenin and E-cadherin were studied; IQGAP1 (a known β-catenin binding protein) was studied in parallel. Immunohistochemistry analysis of paraffin-embedded liver sections was performed essentially as described previously (Javois, L. C. (ed.). 1994. Methods in molecular biology, Vol. 34. Immunocytochemical methods and protocols. Humana Press, Totowa, N.J.) using mouse monoclonal antibodies specific for β-catenin (1:50; clone 14, BD Biosciences, San Jose, Calif.), E-cadherin (1:50; BD Biosciences), IQGAP1 (1:15; BD Biosciences), and IQGAP2 (1:50; Upstate Biotechnology/Millipore, Billerica, Mass.). The detection of primary antibody was carried out using a biotinylated mouse-specific secondary antibody (1:100), streptavidin peroxidase (Rockland Immunochemicals for Research, Gilbertsville, Pa.), and 3,3′-diaminobenzidine as the substrate. Immunohistochemistry analysis of normal wild-type hepatocytes demonstrated membrane-associated expression of E-cadherin, β-catenin, and IQGAP1 (FIG. 11). In contrast, a loss of IQGAP1 expression in plasma membranes and enhanced cytoplasmic expression of IQGAP1 in Iqgap2^{-/-} HCC hepatocytes were evident, and this pattern occurred in parallel with the cytoplasmic translocation and accumulation of β-catenin; loss of E-cadherin expression in plasma membranes was also evident. Although β-catenin expression in Iqgap2^{-/-} hepatocytes of younger mice without HCC was minimally affected, these mice displayed altered IQGAP1 expression, characterized primarily by diffused cytoplasmic staining (FIG. 11).

In an immunoblot analysis, β-catenin bands with a lower than normal (92-kDa) molecular mass were detected exclusively in HCC liver lysates, confirming the presence of previously described mutated forms of β-catenin known to contain somatic activating mutations in mouse and human HCC (FIG. 12A). These mutations prevent β-catenin from being phosphorylated, leading to its cellular redistribution and accumulation in the cytoplasm and/or nuclei (β-catenin activation).

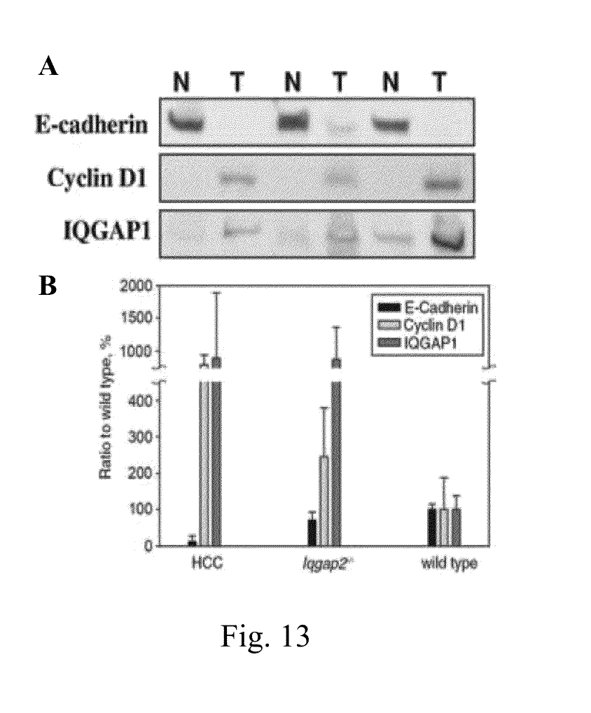

To further investigate the age-dependent activation of the Wnt/β-catenin pathway in Iqgap2^{-/-} livers, liver lysates from 4-month-old wildtype and Iqgap2^{-/-} mice, as well as those from 2-year-old Iqgap2^{-/-} mice affected with HCC, were probed with an antibody specifically recognizing the active form of β-catenin, dephoshorylated on Ser37 or Thr41 (Upstate Biotechnology/Millipore). As shown in FIG. 12B, the dephosphorylated (active) β-catenin was specifically detected in HCC livers and not in samples from younger wild-type or Iqgap2^{-/-} mice (without HCC). Furthermore, this effect was associated with an approximately nine fold increase in cytoplasmic IQGAP1 expression and an approximately eightfold increase in levels of cyclin D1 (FIGS. 13A and B), the cell cycle regulator known to be a downstream target of β-catenin. The expression of E-cadherin in HCC livers was about 12% of that in wild-type livers, consistent with immunohistochemistry data. Noteworthily, more-extensive studies demonstrated enhanced (about 2.5-fold) cyclin D1 and IQGAP1 expression in the livers of younger Iqgap2^{-/-} mice without HCC, establishing that IQGAP1 upregulation and cyclin D1 activation predated histological evidence for HCC development (FIG. 13B).

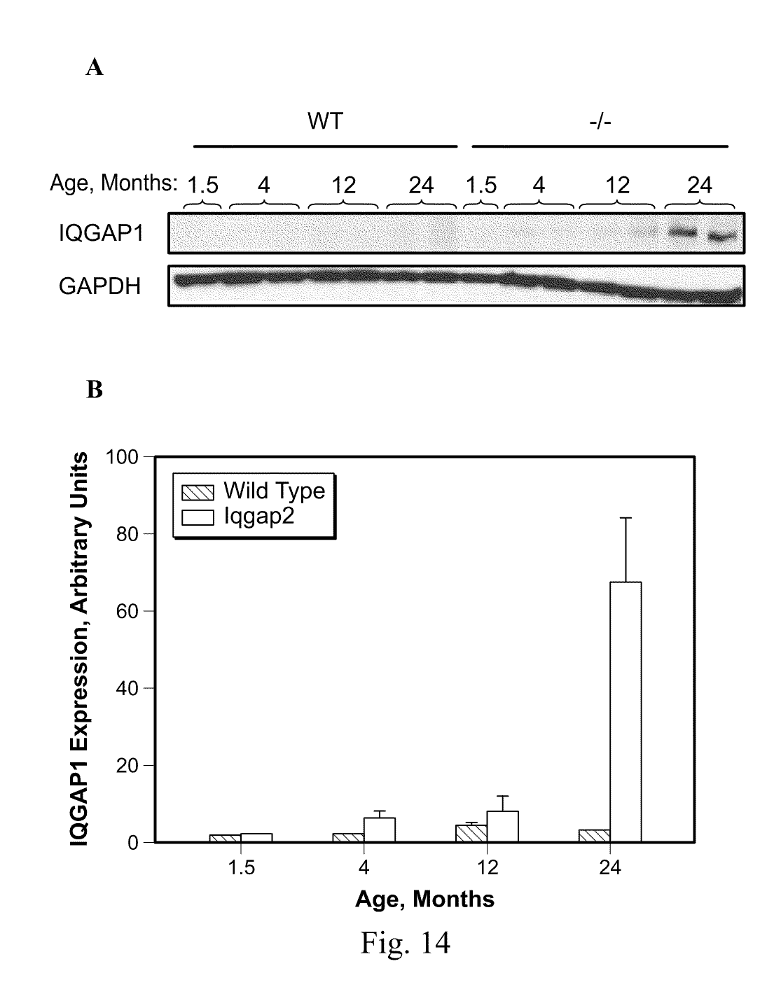

To assess the timing of IQGAP1 upregulation in greater detail, liver lysates from wild-type and Iqgap2^{-/-} mice ranging in age from 1.5 to 24 months were analyzed for IQGAP1 protein expression. As shown in FIGS. 14A and B, IQGAP1 was overexpressed in all Iqgap2^{-/-} samples compared to expression in wild-type samples, irrespective of the age of the mice, and this overexpression thus clearly predated the onset of HCC development. Therefore, while IQGAP1 is evidently upregulated in Iqgap2^{-/-} mice with HCC, the reciprocal induction appears to be present early on (by 16 weeks of age) and is sustained with age. Remarkably, no change in IQGAP1 expression was observed in other organs known to express IQGAP1, including hearts, lungs, kidneys, and spleens, from Iqgap2^{-/-} 4- and 12-monthold mice.

### Example 8

### A Multiprotein β-catenin-E-cadherin-IQGAP1-IQGAP2 Scaffold Exists in Hepatocytes

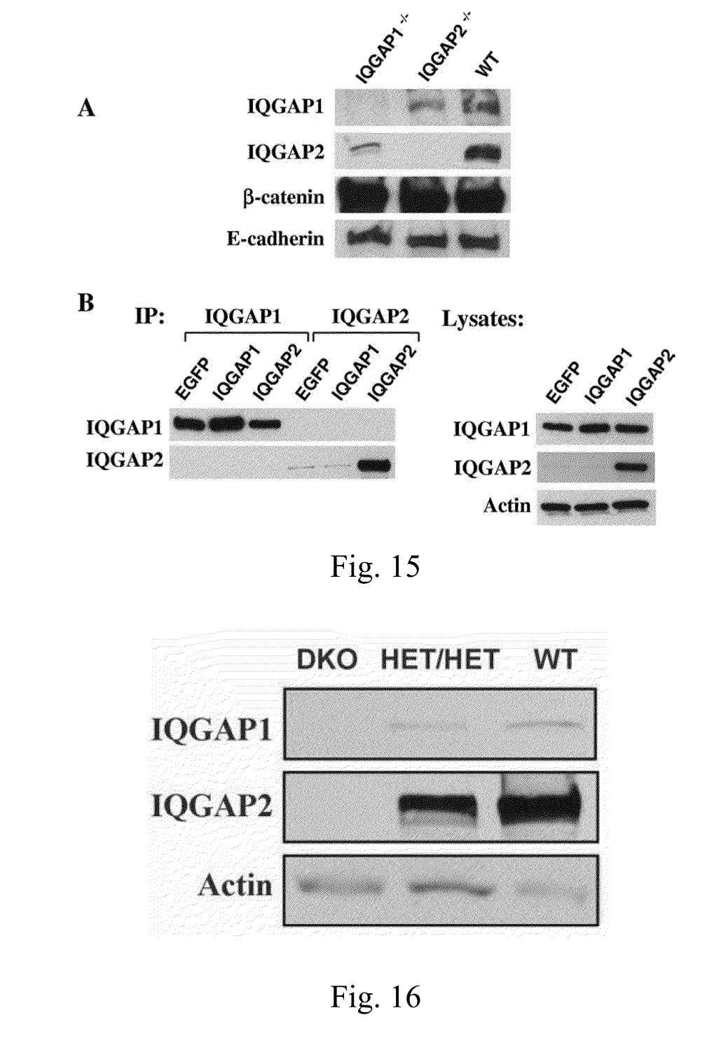

To further probe the relationships between IQGAP1, IQGAP2, β-catenin, and E-cadherin, a series of immunoprecipitation experiments were conducted using whole-liver lysates from Iqgap1^{-/-}, Iqgap2^{-/-}, and wildtype mice. As shown in FIG. 15A, all these proteins are clearly associated as components of a multiprotein scaffolding complex present in normal hepatocytes, as established using a β-catenin pull-down assay. In contrast, there was no evidence for specific interaction between E-cadherin and IQGAP2 in either E-cadherin- or IQGAP2-specific immunoprecipitations. Interestingly, in the β-catenin pull-down assays of Iqgap1^{-/-} and Iqgap2^{-/-} livers, β-catenin was associated with less IQGAP2 and IQGAP1, respectively, than in assays of wild-type livers, suggesting that IQGAP1 and IQGAP2 were functionally cooperative within this complex. Using normal hepatocytes, however, neither IQGAP1- nor IQGAP2-specific pull-down assays coprecipitated the corresponding homologous protein. Transient transfections of COST cells (which endogenously express IQGAP1) were subsequently used as an in vitro model system to specifically address the existence of putative IQGAP1-IQGAP2 heterodimers. As evident in FIG. 15B, IQGAP1 or IQGAP2 were not pulled down by reciprocal immunoprecipitations. Thus, while IQGAP1 and IQGAP2 are normally found as components of a broader quarternary scaffolding complex in hepatocytes, there appears to be no evidence that their presence within this scaffold occurs via direct (heterodimeric) interactions.

Immunoprecipitation experiments were completed using freshly isolated murine livers essentially as described previously (Schmidt, V. A., et al. 2003. Blood 101:3021-3028). In brief, intact livers were homogenized in 25 mM HEPES-0.15 M NaCl buffer, pH 7.45, supplemented with 0.1% Triton X-100 and standard protease inhibitors. After centrifugation at 10,000×g for 15 min at 4° C., 5 to 20 mg of whole-cell lysate was precleared in a 1-ml volume by using 50 μl of protein G agarose (25-μl bed volume; Roche Diagnostics GmbH, Mannheim, Germany) for up to 4 h at 4° C. After centrifugation at 12,000×g for 20 s, supernatants were incubated with 5 μg of protein G agarose-conjugated antibodies overnight at 4° C. Pellets were then collected by centrifugation (12,000×g for 20 s) and washed three times with lysis buffer prior to immunoblotting as outlined above. Alternatively, immunoprecipitation experiments were carried out with COST lysates. COST cells were grown in Dulbecco minimal essential medium supplemented with 10% fetal bovine serum, 100 U/ml of penicillin, and 100 μg/ml of streptomycin sulfate. Transient transfections with full-length human IQGAP1 cDNA (kindly provided by David Sacks, Harvard Medical School) (24) and IQGAP2 cDNA (a gift from Andre Bernards, Harvard Medical School) (8) were completed using Fugene reagent (Roche Diagnostics). Transfection with EGFP-C1 plasmid (Clontech Laboratories, Mountain View, Calif.) served as a control; 24 h posttransfection, cells were washed and solubilized in a buffer containing 25 mM HEPES-0.15 M NaCl buffer, pH 7.45, supplemented with 0.1% Triton X-100 and standard protease inhibitors. Lysates were subsequently used for immunoprecipitation studies as outlined above.

### Example 9

### IQGAP1 Expression is Necessary for Complete Penetrance of the HCC Phenotype

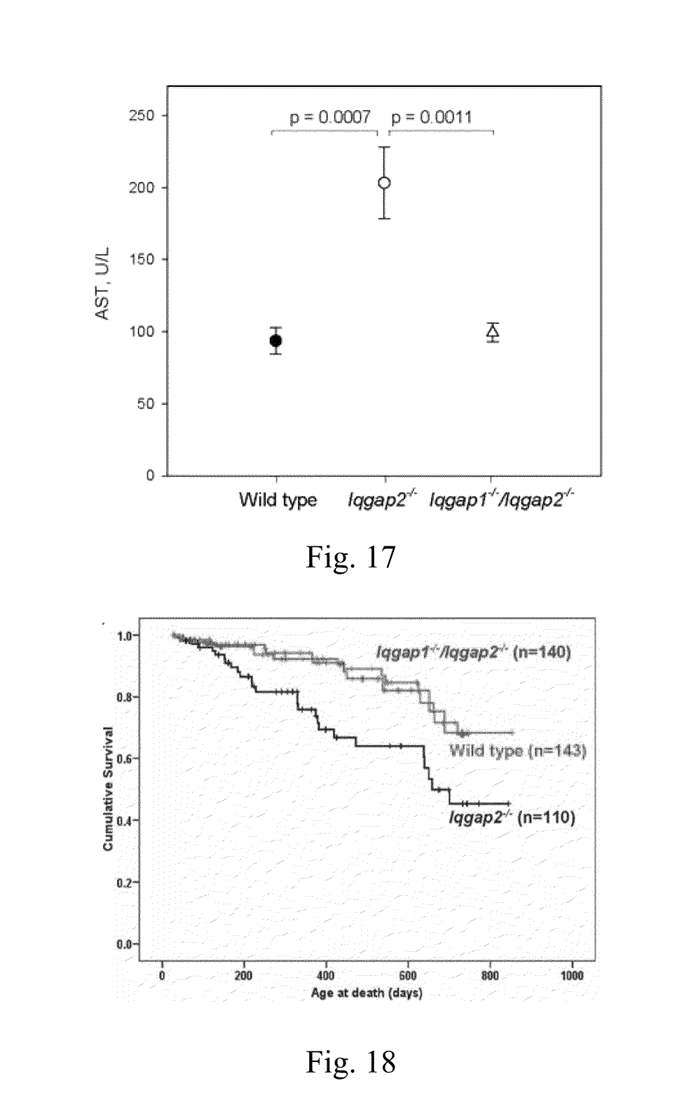

Iqgap1^{-/-}Iqgap2^{-/-} mice were generated for more-detailed analyses, assessing the development of HCC as the primary end point. A lack of hepatic IQGAP1 and IQGAP2 expression was clearly evident by immunoblot analysis, with residual IQGAP1 and IQGAP2 expression in Iqgap1^{+/-}Iqgap2^{+/-} double heterozygotes (FIG. 16). Progressive, age-dependent increases in hepatic AST levels in 18- to 24-month-old Iqgap2^{-/-} mice were demonstrated (compare results in FIG. 16 to FIG. 5A results for 4- and 8-month-old mice), with normal levels found in Iqgap1^{-/-}Iqgap2^{-/-} mice of this age group (FIG. 17). Kaplan-Meier survival curves for mice up to 24 months of age confirmed the progressive increase in the death rate of Iqgap2^{-/-} mice compared to that of wild-type control mice (FIG. 18). Statistically significant (P<0.05) separation of these survival curves was first evident by 200 days after birth, with progressive divergence over time. Entirely consistent with the normalization of the hepatic enzyme defect, Iqgap1^{-/-}Iqgap2^{-/-} mice had survival durations that were essentially identical to those of wild-type controls and were clearly improved compared to those of Iqgap2^{-/-} mice. Kaplan-Meier survival curves and age-dependent HCC incidence data were generated using SPSS software (Statistical Package for the Social Sciences, version 14.0). For all analyses, differences in means between groups were analyzed by a χ² test or two-tailed Student's t test using a P value of 0.05 as a measure of statistical significance.

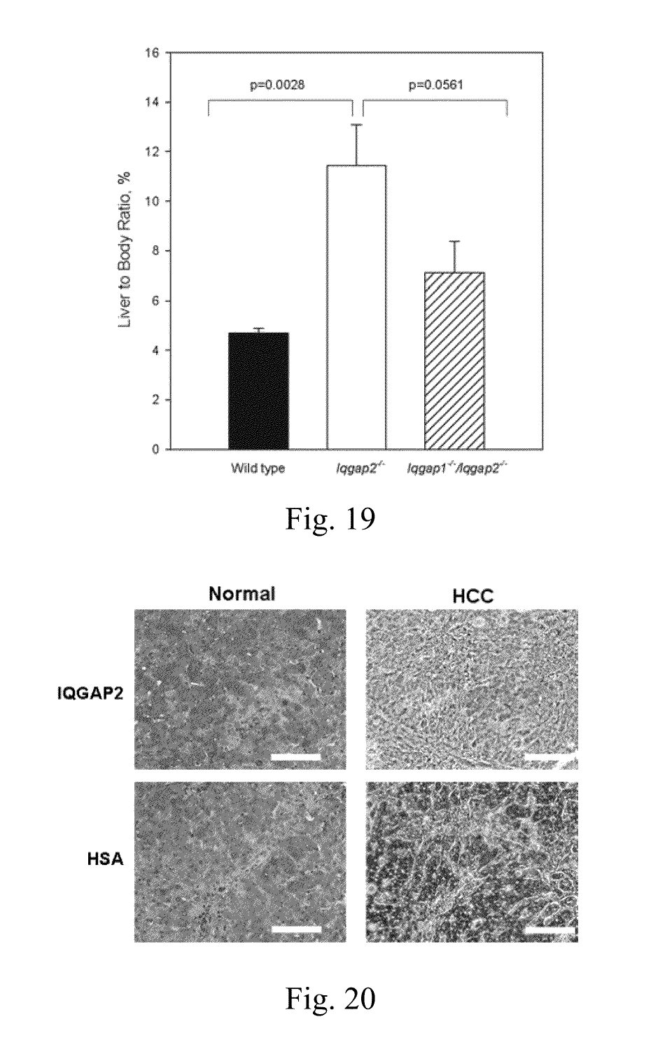

Random necroscopy of Iqgap2^{-/-} mice established that HCC was not found in mice younger than 12 months of age (n=21). To more precisely delineate the incidence and characteristics of HCC in Iqgap2^{-/-} and Iqgap1^{-/-}Iqgap2^{-/-} mice, livers from mice aged 18 to 24 months were isolated. The improved overall survival of Iqgap1^{-/-}Iqgap2^{-/-} mice compared with that of Iqgap2^{-/-} mice occurred in parallel with lower HCC incidence in Iqgap1^{-/-}Iqgap2^{-/-} mice than in Iqgap2^{-/-} mice (as assessed by both macroscopic and microscopic analyses), although HCC still occurred at a frequency greater than that seen in wild-type controls (Table 2). As determined by liver-to-body-weight ratios used as a quantitative parameter for HCC mass, the mean weight of Iqgap2^{-/-} mouse livers affected with HCC was significantly increased compared to that of livers from wild-type controls. Masses of HCCs from Iqgap1^{-/-}Iqgap2^{-/-} mice (when HCCs were identified) were smaller than those of HCCs found in Iqgap2^{-/-} mice (FIG. 19), yet the histological characteristics of the HCCs were similar to those of HCCs from Iqgap2^{-/-} mice. These data suggest that the inactivation of IQGAP1 in mouse liver impairs tumorigenesis caused by IQGAP2 deficiency.

TABLE 2
Incidence of HCC among wild-type, Iqgap2^{-/-}, and Iqgap1^{-/-}/
Iqgap2^{-/-} mice of different ages as established by liver histology

| | <12 months | | | 12-18 months | | | >18 months | | |
|---|---|---|---|---|---|---|---|---|---|
| | | Iqgap^{1-/-}/ | Wild | | Iqgap^{1-/-}/ | Wild | | Iqgap^{1-/-}/ | Wild |
| HCC | Iqgap^{2-/-} | Iqgap^{2-/-} | type | Iqgap^{2-/-} | Iqgap^{2-/-} | type | Iqgap^{2-/-} | Iqgap^{2-/-} | type |
| Present | 0 | 0 | 0 | 1 | 0 | 0 | 18 | 6 | 0 |
| Absent | 21 | 14 | 23 | 6 | 7 | 3 | 3 | 15 | 15 |
| Total | 21 | 14 | 23 | 7 | 7 | 3 | 21 | 21 | 15 |
| Incidence (%) | 0 | 0 | 0 | 14 | 0 | 0 | 86 | 29 | 0 |
| p-value* | N/A | N/A | | 0.49 | N/A | | <0.0001 | 0.023 | |

| | | |
|---|---|---|
| p-value** | 0.299 | 0.0002 |

*Genotype compared to wild type
**Iqgap2^{-/-} vs Iqgap1^{-/-}/Iqgap2^{-/-}

### Example 10

### Immunohistochemical Detection of IQGAP2 in Normal Human Liver and Liver with HCC

Immunohistochemistry studies show diminished IQGAP2 expression in human HCC liver. The evolution of HCC in Iqgap2^{-/-} mice recapitulates that of its human counterpart, proceeding through step-wise phases of hepatocellular apoptosis associated with mitochondrial damage, proliferation with polyploidization, and culminating in HCC development. To address the relevance of our findings in the mouse model to human disease, IQGAP2 expression was analyzed by immunostaining of paraffin-embedded human liver sections. For the pilot study, three individual de-identified normal liver and HCC needle biopsy samples from three different patients, all from the SUNY Stony Brook Tissue Bank collection (Department of Pathology), were sectioned and stained with mouse monoclonal antibodies specific to IQGAP2 (Upstate Biotechnology). In contrast with normal liver, HCC tumors showed only residual expression of IQGAP2 (FIG. 20). Hepatocyte-specific antigen (HSA, abcam, Cambridge, Mass.) antibody served as a positive control.

Lack of IQGAP2 expression was confirmed in 100% (36/36) of formalin-fixed paraffin-embedded human HCC livers studied by immunoistochemistry. All samples also showed a significant overexpression of IQGAP1. Normal livers (13/13), cirrhotic livers (23/23), and benign hepatic adenomas (4/4) showed the reverse, i.e. a high level of IQGAP2 expression and very low levels of IQGAP1

### Example 11

### Transgenic Mouse Overexpressing IQGAP1 in Liver

Transgene constructs are generated by cloning mouse IQGAP1 full length cDNA (GenBank accession number NM_{—}016721) under control of a liver-specific promoter in a plasmid vector based on pBluescipt (Stratagene). Liver-specific promoters include the promoter from albumin, α1-antitrypsin (AAT), phosphoenolpyruvate carboxykinase (PEPCK) and fatty acid binding protein (FABP). An albumin gene enhancer (−10.5-8.5 kb) is added to ensure high levels of targeted expression in hepatocytes (Kellendonk C., et al. Genesis 26:151-135, 2000). Regulatory elements are amplified from mouse liver total RNA by RT-PCR. The transgenic construct also contains an expression tag (such as HA, His or myc).

Transgenic mice are generated as described (see Jensen D R, et al. Am J. Physiol. 273:R683-689, 1997). Briefly, a transgenic construct is microinjected into the fertilized pronuclei of 129J1 wild-type females that have been mated to males of the same genetic background. Founder animals are bred to corresponding wild-type animals, and L-IQGAP1-WT transgenic lines are established. Integration of the transgene in the founder mice and their progeny is determined by Southern blotting and PCR of tail DNA. Nontransgenic littermates are used as controls. Liver-specific transgene expression is confirmed by RT-PCR of liver RNA and immunoblotting of liver lysates. Other tissues are examined as well to ensure liver-specific overexpression.

### Example 12

### Transgenic Mouse Overexpressing IQGAP1 in Liver in the Background of Iqgap^{-/-}/Iqgap2^{-/-} Mouse

The same technical approach described in Example 11 is used except that the IQGAP1 transgenic construct is microinjected into the fertilized pronuclei of Iqgap1^{-/-}/Iqgap2^{-/-} double knockout females.

### Example 13

### Liver-Specific IQGAP1 Knockout Mouse in the Background of Iqgap2^{-/-} Mouse

To generate a liver-specific IQGAP1 knockout (L-IQGAP1-KO-Iqgap2) in the background of Iqgap2^{-/-} mouse, the targeting construct used for the production of the conventional IQGAP1 knockout model described in (Li S., et al. Mol Cell Biol 20:697-701, 2000) is modified to include loxP sites and a liver-specific promoter. L-IQGAP1-KO-Iqgap2^{-/-} mice are generated using the Cre/loxP gene targeting strategy as described previously (She P., et al., Mol Cel Biol 20:6508-6517, 2000). The targeting construct is microinjected into the fertilized pronuclei of Iqgap2^{-/-} females. Lack of hepatic IQGAP1 expression is confirmed on both RNA and protein levels as described above. In contrast to Iqgap1^{-/-}/Iqgap2^{-/-} double knockout model, L-IQGAP1-KO-Iqgap2^{-/-} mice allow differentiating the effects of IQGAP1 and IQGAP2 deficiency in one isolated organ, the liver.

### Example 14

### In Vivo Knockdown of IQGAP2 Gene in Mouse Liver Using siRNA

The full length mouse IQGAP2 mRNA transcript sequence is publicly available at GenBank (accession number NM_{—}027711). Double-stranded siRNAs (21-22 nucleotides in length) specific to various target regions of the IQGAP2 transcript is synthesized. Mouse IQGAP2 gene contains 34 exons and any of them can be targeted with siRNA to disrupt gene function.

siRNAs is cloned into pSilencer plasmid (available from Ambion, Austin, Tex.) under control of CMV promoter to create siRNA-expressing constructs for expression in mammalian cells. Several approaches for siRNA delivery into mouse models have been developed recently: 1) “Naked” siRNAs can be delivered by intravenous injection (via tail vein or portal vein) or direct injection into the liver; 2) siRNA also can be incorporated into liposomes for efficient intravenous or intraperitoneal delivery; 3) liver-specific delivery of siRNA can also be efficiently achieved using recombinant viral vectors (adenovirus, adeno-associated virus, AAV, and lentivirus). For viral vector delivery, siRNA of choice is subcloned into appropriate viral expression cassettes and used for virus generation. siRNA-containing virus is then injected into a mouse. For review see Kawakami S, et al Drug Metab Pharmacokinet 22:142-151, 2007; De Paula D, et al RNA 13:431-456, 2007; Cruz P E, et al Lab Invest 87:893-902, 2007; Lu W, et al Cell 119:97-108, 2004.

### Example 15

### Down Regulation of IQGAP1 or IQGAP2 in a Human Liver Hepatocellular Cell Line

Either IQGAP1 or IQGAP2 expression in human hepatocellular carcinoma HepG2 cell line was downregulated using IQGAP1 or IQGAP2 specific siRNA. The siRNAs (Ambion) are as follows: GAP1 siRNA: sense (5′-3′): GGUUGACUUCACAGAAGAAtt (SEQ ID O:21), antisense: UUCUUCUGUGAAGUCAACCtt (SEQ ID NO:22); GAP2 siRNA: sense (5′-3′); GGAAUUCAGGAAAUAUUUCtt (SEQ ID NO:23), antisense: GAAAUAUUUCCUGAAUUCCtg (SEQ ID NO:24).

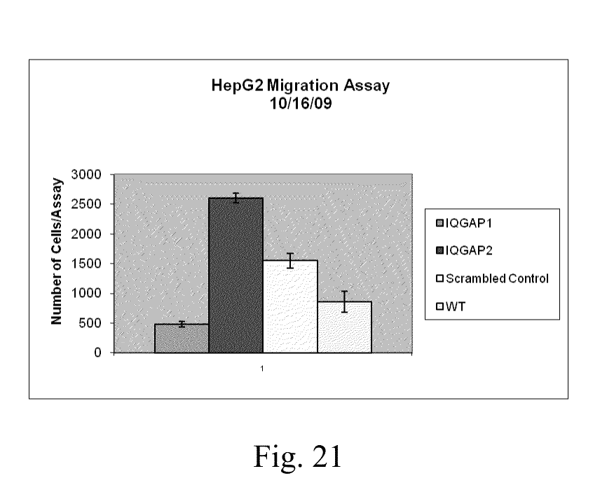

24 hours before transfection, test cells were seeded in 12-well plates in an amount to yield 30% to 50% confluence at the time of assay. For each assay, 6 μl of an interfering RNA (siRNA) stock solution (50 μM) was diluted in 50 μl of Opti-MEMI medium without FBS. 1 μl of Lipofectamine RNAiMAX was added to the Opti-MEMI/SiRNA solution. The solution was mixed gently and incubated for 10-20 min. at room temperature. For transfection, media was aspirated from the test cells, which were washed with PBS. The test cells were then incubated in 600 μl of DMEM with 5.0% FBS (without Pen/Strep) per well. Opti-MEMI/siRNA/Lipofectamine RNAiMAX solutions (57 μl) were added to the test cells (10 nM final siRNA concentration). 48 hours later cell migratory ability was measured by a migration assay. (FIG. 21).

The downregulation of IQGAP2 protein expression results in the increased cell migration, consistent with the proposed role of IQGAP2 as a tumor suppressor. The downregulation of IQGAP1 expression had an opposite effect on the cells migratory ability.

We claim:

1. A non-human mammal, which comprises a targeted gene disruption in an endogenous Iqgap2 gene.

2. The non-human mammal of claim 1, which is homozygous for the targeted gene disruption.

3. A non-human mammal, which comprises a transgene that encodes an RNA that inhibits expression of an endogenous Iqgap2 gene.

4. The non-human mammal of claim 3, wherein the RNA is selected from the group consisting of an antisense RNA, and a siRNA.

5. A non-human mammal, which comprises a transgene that overexpresses an Iqgap1 gene.

6. The non-human mammal of claims 1 to 4, which comprises a null allele of the endogenous Iqgap1 gene.

7. The non-human mammal of claims 1 to 4, which is homozygous for a null allele of the endogenous Iqgap1 gene.

8. The non-human mammal of claim 6 or 7, wherein the null allele is a targeted disruption in an endogenous Iqgap1 gene.

9. The non-human mammal of any one of claims 1 to 5, which exhibits an age-dependent increase in apoptosis of hepatocytes.

10. The non-human mammal of any one of claims 1 to 5, which exhibits an age-dependent increase in hepatocellular carcinoma.

11. The non-human mammal of any one of claims 6 to 10, which exhibits a reduction in age-dependent apoptosis or hepatocellular carcinoma associated with reduced Iqgap2 levels.

12. The transgenic non-human mammal of any one of claims 1 to 11, wherein the transgenic non-human mammal is a mouse.

13. A cell isolated from the transgenic non-human mammal of any one of claims 1 to 12.

14. The isolated cell of claim 13, which is a hepatocyte, fibroblast, myocyte, adipocyte, lymphocyte, megacaryocyte, or platelet.

15. The cell of claim 13 or 14, which overexpresses Iqgap1.

16. A cell line derived from the non-human mammal of any of claims 1 to 12.

17. A cell line which comprises an inactivated endogenous Iqgap2 gene.

18. The cell line of claim 17, which is homozygous for the inactivated endogenous Iqgap2 gene.

19. The cell line of claim 17, wherein the endogenous Iqgap2 gene is inactivated by a targeted disruption.

20. The cell line of claim 17, wherein the endogenous Iqgap2 gene is inactivated by a small interfering RNA, a microRNA, or an anti-sense RNA.

21. The cell line of any one of claims 17 to 20, which further comprises an inactivated Iqgap1 gene.

22. The cell line of any one of claims 17 to 20, which is engineered to overexpress Iqgap1.

23. The cell line of claim 21, wherein the genome of the cell line comprises a null allele of the endogenous Iqgap1 gene.

24. The cell line of claim 17 wherein the cell line is an embryonic stem cell line.

25. A targeting construct comprising a first polynucleotide sequence homologous to a first portion of an endogenous Iqgap2 gene, a second polynucleotide sequence homologous to a second portion of the endogenous Iqgap2 gene, and a selectable marker located between the first and second polynucleotide sequences.

26. The targeting construct of claim 25, wherein the Iqgap2 gene is a mouse gene.

27. A method of producing a transgenic non-human mammal comprising a disruption in an endogenous Iqgap2 gene, comprising:
a) introducing a targeting construct capable of disrupting the endogenous Iqgap2 gene into an embryonic stem cell of the mammal;
b) selecting an embryonic stem cell that has undergone homologous recombination
c) introducing the recombinant embryonic stem cell into a blastocyst;
d) implanting the blastocyst into the mammal, wherein the mammal gives birth to a chimeric mammal; and
e) breeding the chimeric mammal to produce the transgenic mammal,
wherein when the disruption is homozygous, the transgenic mammal lacks production of a functional IQGAP2 protein.

28. The method of claim 27, wherein the Iqgap1 gene is overexpressed in hepatocytes of the transgenic mammal

29. A method of identifying a compound as a therapeutic agent for the treatment of hepatocellular carcinoma, comprising:
contacting the compound with a test cell that underexpresses Iqgap2, wherein Iqgap1 is overexpressed, and
identifying the compound as a therapeutic agent for treatment of hepatocellular carcinoma if IQGAP1 activity is reduced in response to the compound.

30. The method of claim 29, wherein Iqgap1 is overexpressed in response to Iqgap2 underexpression.

31. The method of claim 29, wherein the test cell is engineered to overexpress Iqgap1.

32. A method of identifying a compound as a therapeutic agent for the treatment of hepatocellular carcinoma, comprising:
contacting the compound with a test cell that overexpresses Iqgap1, and
identifying the compound as a therapeutic agent for treatment of hepatocellular carcinoma if IQGAP1 activity is reduced in response to the compound.

33. The method of claim 29 or 32, wherein the test cell is a hepatocyte.

34. The method of claim 29 or 32, wherein the IQGAP1 activity is evaluated by observing the level of Iqgap1 expression using an Iqgap1 reporter gene construct.

35. A method of identifying a compound as a therapeutic agent for the treatment of hepatocellular carcinoma, comprising:
providing a non-human mammal which comprises a targeted gene disruption in an endogenous Iqgap2 gene and exhibits an age-dependent increase in apoptosis of hepatocytes;
administering the compound to the non-human mammal, and
identifying the compound as a therapeutic agent if it reduces the age-dependent increase in apoptosis.

36. A method of identifying a compound as a therapeutic agent for the treatment of hepatocellular carcinoma, comprising:
providing a non-human mammal which comprises a targeted gene disruption in an endogenous Iqgap2 gene and exhibits an age-dependent increase in hepatocellular carcinoma;
administering the compound to the non-human mammal, and
identifying the compound as a therapeutic agent if it reduces the age-dependent increase in hepatocellular carcinoma.

37. A model of human hepatocellular carcinoma comprising hepatocytes in which Iqgap2 is underexpressed.

38. The model of claim 37, wherein the reduction in the expression of Iqgap2 is accomplished by creating a null allele of the endogenous Iqgap2 gene.

39. The model of claim 37, wherein the null allele of the endogenous Iqgap2 is homozygous.

40. A method of treating or preventing hepatocellular carcinoma comprising administering an agent that decreases Iqgap1 function in hepatocytes.

41. The method of claim 40, wherein the agent that decreases Iqgap1 function is an siRNA.

42. The method of claim 40, wherein the agent that decreases Iqgap1 function is a synthetic blocking peptide.

43. The method of claim 42, wherein synthetic blocking peptide is directed against IQGAP1 domains selected from the group consisting of: actin-binding calponin homology domain, SH3-mimicking domain, calmodulin-binding domains, and GTPase-binding domain.

44. A method of treating or preventing hepatocellular carcinoma by administering an agent that enhances Iqgap2 function in hepatocytes.

45. The method of claim 44, wherein the agent comprises a vector that expresses Iqgap2.

46. The method of claim 45, wherein the Iqgap2 is expressed from a liver-specific promoter.

47. The method of claim 46, wherein the liver-specific promoter is selected from the group consisting of promoter from the genes: albumin, α1-antitrypsin (AAT), phosphoenolpyruvate carboxykinase (PEPCK) and fatty acid binding protein (FABP).

48. The method of claim 44, wherein the agent comprises a methylation inhibitor.

49. The method of claim 48, wherein the methylation inhibitor is a nucleoside inhibitor.

50. The method of claim 49, wherein the nucleoside inhibitor is selected from the group consisting of 5-azacytidine, 5-aza-2′-deoxy-cytidine, zebularine, and 5-fluoro-2′-deoxycytidine.

51. The method of claim 48, wherein the methylation inhibitor is a non-nucleoside inhibitor.

52. The method of claim 48, wherein the non-nucleoside inhibitor is selected from the group consisting of procainamide, and hydralazine.
